(19) United States
(12) Patent Application Publication
(10) Pub. No.: US 2011/0256096 A1
(43) Pub. Date: Oct. 20, 2011

(54) Estrogen receptor ligand and/or interferon beta treatment for neurodegenerative diseases

(76) Inventors: Rhonda R. Voskuhl, Los Angeles, CA (US); Seema K. Tiwari-Woodruff, Sherman Oaks, CA (US)

(21) Appl. No.: 12/803,873

(22) Filed: Jul. 8, 2010

Related U.S. Application Data
(60) Provisional application No. 61/270,492, filed on Jul. 8, 2009.

(51) Int. Cl. A61K 38/21; A61P 29/00; A61P 25/00; A61P 25/28; A61K 31/565; A61K 31/277

(52) U.S. Cl. 424/85.6; 514/182; 514/520

(57) ABSTRACT

This invention relates generally to novel treatments to prevent neurodegeneration in the central nervous system comprising a therapeutic dosage of an estrogen receptor ligand and/or an immunotherapeutic compound, such as beta-interferon, to ameliorate the effects of the neurodegenerative disease and to stimulate repair.

### PRIORITY INFORMATION

This application claims priority from U.S. Provisional Patent Application No. 61/270,492, filed Jul. 8, 2009.

This invention was made with Government support of Grant No. NS062117, awarded by the National Institutes of Health. The Government has certain rights in this invention.

### BACKGROUND OF THE INVENTION

1. Field of the Invention

This invention relates generally to a novel treatment to prevent neurodegeneration in the central nervous system due to diseases such as multiple sclerosis (MS), Alzheimer's disease, Parkinson's disease, spinal cord injury, stroke, etc. More specifically, the present invention relates to treatments comprising a therapeutic dosage of an estrogen receptor ligand and/or an immunotherapeutic compound, such as beta-interferon, to ameliorate the effects of the neurodegenerative disease and to stimulate repair.

2. General Background

This application incorporates by reference PCT Application No PCT/08/012353, published as WO/2010/050916.

Neurodegenerative diseases of the central nervous system (CNS) are characterized by the loss of neuronal and glial components and functionality. Therapeutic strategies that induce effective neuroprotection and enhance intrinsic repair mechanisms are central goals for future therapy of neurodegenerative diseases.

Demyelinating diseases, such as multiple sclerosis, are characterized by inflammatory demyelination and neurodegeneration of the CNS. Despite the ability of the adult brain to generate oligodendrocytes (OL) with myelination capacity, remyelination in experimental autoimmune encephalomyelitis (EAE), the animal model for multiple sclerosis (MS), is incomplete. Current anti-inflammatory or immunomodulatory treatments, while partially effective in the relapsing stage of the disease, have only modest to minimal effects on the development of neurodegeneration and clinical disability in the secondary progressive phase of disease. Therefore, it is important to find novel treatments which could prevent demyelination and/or enhance remyelination.

The rationale for almost all therapies for MS to date has been to reduce inflammation. Immunomodulatory therapies, such as interferon-β, glatiramer acetate, and mitoxantrone have considerably improved the therapeutic options for patients with MS. These agents reduce relapse rates and reduce appearance of MRI enhancing lesions. However, their efficacy in preventing accumulation of disability and their impact on disease progression has been disappointing. Identifying a drug that stimulates endogenous myelination and spares axon degeneration would theoretically reduce the rate of disease progression.

The currently immunomodulatory treatments available for MS reduce relapses by one third to one half They are given by subcutaneous injection either every day (Copaxone) or three times a week (Rebif, Betaseron) or by intramuscular injection once a week (Avonex). Other more aggressive treatments are given less frequently by intravenous infusion (Novantrone, Tysabri), but they are associated with very serious life threatening adverse events. Of the list of relatively safe treatments (Copaxone, Rebif, Betaseron, Avonex), many patients prefer the once a week regimen of interferon beta (Avonex), but unfortunately this dose has been shown to be relatively “low” and associated with less efficacy as compared to higher interferon beta doses with more frequent treatment regimens. Thus, combinations of immunomodulatory agents with other effective agents are desirable so as to minimize the risks and improve the efficacy of current therapies.

There are no current neuroprotective drugs that can be taken for long durations of time without significant side effects. Estrogens, as well as the use of estrogen receptor (ER) alpha ligand treatments, have been studied in disease and injury animal models and in humans. Estrogen, and estrogen receptor alpha ligand treatments, are effective in some disease and injury models. For example, they are both anti-inflammatory and neuroprotective in EAE, and there is a dose response whereby higher levels are more protective. However, in humans, treatment with estrogens or ER alpha ligands may not be tolerable due to the induction of breast cancer and uterine cancer, which are mediated by estrogen receptor alpha in the breast and uterus, respectively. The risk:benefit ratio of any estrogen treatment must be considered for use in neurodegenerative diseases. Estrogens in the form of hormone replacement therapy have been associated with side effects and therefore are not recommended for use in healthy menopausal women. While the risk:benefit ratio in debilitating neurodegenerative diseases is clearly different than the risk:benefit ratio in healthy individuals, optimizing efficacy and minimizing toxicity, remains the goal. Hence, determining which estrogen receptor mediates the neuroprotective effect of estrogen treatment is of central importance.

Investigations in EAE have also shown differential effects of estrogen receptor (ER) a ligand treatment, which reduced CNS inflammation versus ERβ ligand treatment, which preserved axon and myelin despite having no effect on CNS inflammation in spinal cords. Despite the fact ERβ has been shown to be expressed widely in the CNS in adult mice, in most neurological disease models, the protective effect of estrogen treatment has been shown to be mediated through ERα and has been associated with anti-inflammatory effects. Nonetheless, further investigation of ERβ ligands to prevent demyelination and/or enhance remyelination are warranted. This is of interest for example, for the treatment of MS, since inefficiency or failure of myelin-forming OLs to remyelinate axons and preserve axonal integrity remains a major impediment in the repair of MS lesions and is principally responsible for axonal and neuronal degeneration leading to chronic disability. Further, estrogen receptor beta (ERβ) is not associated with breast or uterine cancer. The ligand has no known toxicity or blood brain barrier permeability issues. Thus, estrogen receptor beta ligands may be used for long durations and/or for high risk patients who could not otherwise tolerate estrogen or estrogen receptor alpha ligand treatment.

For diseases that do not appear to have an inflammatory component, but only a neurodegenerative component, then the estrogen receptor beta ligand treatment alone may also be useful. Notably, the role of inflammation in Alzheimer's disease, Parkinson's disease, brain or spinal cord injury and stroke are primarily purely neurodegenerative diseases or injuries, but there may be a minor inflammatory component. To date, for Alzheimer's disease, for example, there are only treatments that can be used in short term duration. Thus, alternative treatments are desirable.

Presently, the only previously described neuroprotective agent for EAE, which did not decrease CNS inflammation, were blockers of glutamate receptors. These treatments resulted in a modest reduction in neurologic impairment and the effect was lost after cessation of treatment. Glutamate blockers are currently used in amyotrophic lateral sclerosis (ALS) and Alzheimer's disease with modest success. In MS, brain atrophy on MRI has been detected at the early stages of disease, thus a neuroprotective agent would need to be started relatively early, generally at ages 20-40 years, and continued for decades. Since glutamate is needed for normal neuronal plasticity and memory, treatment of relatively young individuals with glutamate blockers for decades may be associated with significant toxicity.

Hence, the identification of an alternative neuroprotective agent represents an important advance in preclinical drug development in MS and other chronic neurodegenerative diseases or injuries.

### INVENTION SUMMARY

The present invention is directed to a medicament or treatment to prevent neurodegeneration in the central nervous system due to neurodegenerative diseases, such as MS, Parkinson's disease, cerebellar ataxia, Down's Syndrome, epilepsy, strokes, Alzheimer's disease, and brain and/or spinal cord (CNS) injury.

In accordance with one embodiment of the present invention, a method for treating the symptoms of a neurodegenerative disease in a mammal is provided, the method comprising the administration of an estrogen receptor beta (“ERβ”) ligand and/or an anti-inflammatory, such as a Type 1 interferon (such as interferon beta (IFN-β)). In one aspect of the invention the combination of an ERβ ligand and a Type 1 interferon may be additive or synergistic. At least one advantage of this invention is to reduce the dosage of β interferon to patients, which causes flu-like symptoms.

In accordance with another embodiment of the present invention, the invention comprises the use of a ERβ ligand to effectuate a neuroprotective effect. In one embodiment ERβ may be used to delay the onset or progression of disease or injury after the acute phase and/or decrease ameliorate neurodegeneration, and the clinical symptoms thereof.

In accordance with another embodiment of the present invention, the invention comprises the use of a ERβ ligand to effectuate a repair effect within the nervous system. In one embodiment ERβ may be used to maintain myelination or promote myelination in the nervous system, and the clinical symptoms thereof.

For example, treatment with a therapeutically effective dosage of ERβ ligand may result in: fewer demyelinated and/or damaged axons; enhanced oligodendrocyte differentiation; more myelinated axons, including axons with intact nodes of Ranvier; an increase in mature oligodendrocyte numbers; an increase in myelin sheath thickness; and/or enhanced axon transport. Treatment with a therapeutically effective dosage of ERβ ligand may consequently result in improved clinical scores for mammal experiencing a neurodegenerative condition, including in the presence of inflammation.

In accordance with another embodiment of the invention, a anti-inflammatory agent may be used alone or in combination with a neuroprotective agent to treat a neurodegenerative condition.

For example, treatment with a combination of INFβ and ERβ ligand may be superior to INFβ with respect to ameliorating clinical disability, and reducing neuropathology, in MS for example. INFβ and ERβ ligand may act synergistically to decrease levels of IL17 from autoantigen stimulated peripheral immune cells and by decreasing VLA-4 expression on CD4+ T lymphocytes. Further, a lower dose of INFβ ligand may be utilized to effectuate anti-inflammatory benefits of such treatments. One advantage of the invention may include that the combination of INFβ and ERβ ligand may permit weekly dosing of the interferon, for example, and maintenance of the minimal adverse event profile of the relatively low dose interferon.

In one embodiment, the ERβ ligand may include, diarylpropionitrile (“DPN”) at a dose of about 2-16 mg/kg/day, or about 4-12 mg/kg/day, or about 8 mg/kg/day. Other ERβ ligand may be selected, such estriol (at a dose of about 2-16 mg/kg/day, or about 4-12 mg/kg/day, or about 8 mg/kg/day).

In one embodiment, the beta interferon may be interferon-β 1a or interferon-β 1b, such as Rebif, Betaseron, or Avonex, or the active ingredients therein. The dosages of each of these currently used are: Avonex-interferon beta-1a, 30 mcg, injected intramuscularly, once a week; Rebif-interferon beta-1a, 44 or 22 mcg, injected subcutaneously, three times per week; Betaseron-interferon beta-1b, 0.25 mg, injected subcutaneously, every other day. The dosage of beta interferon useful in this invention may include lower doses than generally used, for example, Avonex at about 15-29 mcg, Rebif at about 11-21 mcg, Betaseron at about 0.125-0.24 mg. Alternatively, a patient may achieve a greater clinical benefit using a dosage at or about the currently approved interferon dose, but adding treatment with estrogen receptor beta ligand. In one embodiment, the beta interferon may include, for example, a dose of Avonex at about 30 mcg, Rebif at about 22-44 mcg, Betaseron at about 0.25 mg.

The above described and many other features and attendant advantages of the present inventions will become apparent from a consideration of the following detailed description when considered in conjunction with the accompanying examples.

### BRIEF DESCRIPTION OF THE DRAWINGS

FIG. 1 is a graph depicting mean clinical scores±SD over time of EAE mice treated with vehicle (black triangle), IFNβ (red diamond), ERβ ligand (orange inverted triangle), or the combination (green square) (p<0.0001).

FIG. 2 are 40× images of the lateral funiculus of the thoracic spinal cord of day 40 (top) and day 70 (bottom) EAE mice stained for myelin with MBP; and plots depicting axonal densities between treatment groups at day 40 (left) and day 70 (right).

FIG. 3 are 10× images of the lateral funiculus of the thoracic spinal cord of day 40 EAE mice were stained for the pan-immune cell marker CD45 (top), Mac3 (middle), and CD3 (bottom); and plots depicting quantification of cells stained for CD45⁺ between treatment groups.

FIG. 4 are plots depicting quantification of Th1, Th2 and Th17 cytokine levels measured from supernatants of splenocytes stimulated with MOG 35-55 between treatment groups: IL-10 (4A, p<0.0001), IL-4 (4B, p=0.001), IFNγ (4C, p<0.0001), TNFα (4D, p=0.0001), and IL-12p70 (4E, p=0.001).

FIG. 5 are plots depicting MMP-9 levels in supernatants between treatment groups.

FIG. 6 are representative histograms of the level of VLA-4 expression on gated CD4 and CD8 (T cells), CD19 (B cells), and CD11b (macrophages and monocytes).

FIG. 7-I(A) is a graph depicting mean clinical scores±SD over time of EAE mice treated with vehicle (red triangle), ERβ ligand (blue circle), or normal animals (black square) (p<0.001); (B) are representative plates 29-48 of the Franklin and Paxinos atlas; (C) are representative histological sections demonstrating the level of infiltrating cells (represented by DAPT⁺ cells) after induction of EAE by treatment group; FIG. 7-II (A) is a graph depicting mean clinical scores±SD over time of EAE in intact (gray triangle) and ovariectomized (red square) mice, as well as intact (black triangle) and ovariectomized normal (red circle) mice; (B) are histological slices showing staining for PLP_EGFP (top) and MBP(bottom) in intact noral and intact EAE mice; (C) is a graph depicting mean clinical scores±SD over time in intact normal (black square), intact EAE+vehicle (red triangle) and EAE+ERβ ligand treated (blue square) mice; (D) is a representative cross section of spinal cord depicting the locations of sections shown in panel (E); (E) are histological spinal cord sections showing MBP+DAPI (top) and NF200 (bottom).

FIG. 8-I (A) are representative histological sections stained for CD45, Mac3, CD3 and GFAP+DAPI for each treatment group; (B) are bar graphs depicting the quantification of CD45, Mac3, CD3 and GFAP intensity for each treatment group; FIG. 8-II (A) are are histological sections stained for PLP_EGFP+CD45, MBP+(DAPI) and NF200 for each treatment group; (B) are bar graphs depicting the quantification of MBP intensity and NF200 axons for each treatment group in intact and ovariectomized mice.

FIG. 9(A) are representative histological sections stained for PLP_EGFP, PLP_EGFP+DAPI, olig2+DAPI, GST+DAPI and PDGFRα+DAPI for each treatment group; (B) are bar graphs depicting a quantification of stained cells as shown (A).

FIG. 10(A) are representative histological sections stained for each treatment group; (B) is a bar graph depicting normalized MBP intensity.

FIGS. 11(A) and (B) are representative electron micrographs from each treatment group; (C) (i) and (ii) are bar graphs depicting myelin thickness and g ratios for each treatment group and (iii) and (iv) are scatter plots of axon diameter vs. g ratio and axon diameter vs. myelin thickness for each treatment group.

FIGS. 12(A) and (B) are representative confocal images from representative histological sections stained for NF200+MBP (A) or (B) β-APP; (C) is bar graph depicting quantification of β-APP intensity for each treatment group.

FIGS. 13(A) and (C) are representative histological sections stained for Caspr+Nav1.6 and Kv1.2 for each treatment group; (B) are bar graphs depicting a quantification of Caspr protein pairs alone (top) or encompassing Nav1.6 protein.

FIG. 14(A) are representative slices from the corpus collosum (“CC”) from which compound action potential (CAP) responses were recorded; (B) are typical CC CAPs from normal (black-top), EAE+vehicle (red bottom) and EAE+ERβ ligand (blue middle) mice; (C) and (D) are graphs depicting the quantifications of N1 and N2 CAP amplitudes in each treatment group at early and late time points.

FIG. 15(A) are example waveforms for each treatment group; (B) is a graph depicting average C2/C1 ratios vs. interphase intervals for each treatment group.

FIGS. 16(A) and (C) are representative histological sections stained for PLP_EGFP+dextran red for each treatment group; (B) and (D) are bar graphs depicting the quantification of DR and NF200 intensity for each treatment group;

### DETAILED DESCRIPTION

This description is not to be taken in a limiting sense, but is made merely for the purpose of illustrating the general principles of the invention. The section titles and overall organization of the present detailed description are for the purpose of convenience only and are not intended to limit the present invention.

Generally, the invention involves a method of treating a mammal exhibiting clinical symptoms of a neurodegenerative condition comprising administering a therapeutically effective dosage of at least one of a ERβ ligand and/or a Type I interferon, such as INFβ to effectuate a neuroprotective, repair and/or anti-immune effect, and thus the clinical condition of the mammal.

The beneficial effect of treatment can be evidenced by a protective effect on the progression of disease symptomology, a reduction in the severity and/or improvement in of some or all of the clinical symptoms, or an improvement in the overall health of the subject.

For example, patients who have clinical symptoms of a neurodegenerative condition often suffer from a variety of symptoms. MS patients, for example, suffer from the following symptoms: weakness, numbness, tingling, loss of vision, memory difficulty and extreme fatigue. Thus, an amelioration of disease in MS would include a reduction in the frequency or severity of onset of weakness, numbness, tingling, loss of vision, memory difficulty and extreme fatigue. On imaging of the brain (MRI) amelioration or reduced progression of disease would be evidenced by a decrease in the number or volume of gadolinium enhancing lesions, a stabilization or slowing of the accumulation of T2 lesions and/or a slowing in the rate of atrophy formation. Immunologically, an increase in Th2 cytokines (such as IL-10) a decrease in Th1 cytokines (such as interferon gamma) are generally associated with disease amelioration.

Patients may also express criteria indicating they are at risk for neurodegenerative conditions. These patients may be preventatively treated to delay the onset of clinical symptomology. More specifically, patients who present initially with clinically isolated syndromes (CIS) may be treated using the treatment paradigm outlined. These patients have had at least one clinical event consistent with MS, but have not met full criteria for MS diagnosis since the definite diagnosis requires more than one clinical event at another time. Treatments of the present invention could be advantageous at least in providing a protective or reparative effect after the acute phase of clinically definite MS.

ERβ Ligands. One agent useful in this invention alone or in combination is an ERβ ligand, which may be steroidal or non-steroidal agents which bind to and/or cause a change in activity or binding of the estrogen receptor β. In one embodiment, an ERβ agonist useful in this invention may be the steroid estriol or the non-steroidal analog diarylpropionitrile (“DPN”). Additionally, analogues of ERβ ligands that are more selective for ERβ than ERα receptor, which are know, to those skilled in the art, may also be useful in the present invention. For example, ERβ agonists which are analogs to DPN are known in the art (Harrington, W R et al., “Activities of estrogen receptor alpha- and beta-selective ligands at diverse estrogen responsive gene sites mediating transactivation or transrepression,” Molecular and Cellular Endocrinology, 29 Aug. 2003, vol. 206(1-2), pp. 12-22; Meyers, M J et al., “Estrogen receptor-beta potency-selective ligands: structure-activity relationship studies of diarylpropionitiles and their acetylene and polar analogues,” Journal of Medicinal Chemistry, 22 Nov. 2001, vol. 44(24), pp. 4230-4251).

By way of example only, in one embodiment, the ERβ ligand may include, diarylpropionitrile (“DPN”) at a dose of about 2-16 mg/kg/day, or about 4-12 mg/kg/day, or about 8 mg/kg/day. Other ERβ ligand may be selected, such estriol (at a dose of about 2-16 mg/kg/day, or about 4-12 mg/kg/day, or about 8 mg/kg/day), or other estriol ligands as those described in “Estrogen Receptor-β Potency-Selective Ligands: Structure—Activity Relationship Studies of Diarylpropionitriles and Their Acetylene and Polar Analogues” Marvin J. Meyers, et al., J. Med. Chem., 2001, 44 (24), pp 4230-4251, which is incorporated herein by reference. One of skill in the art would be able to determine the dosage of an alternative ERβ ligand by known dose response techniques.

Type 1 interferons. Type 1 interferons may be used alone or in combination with an ERβ ligand to achieve the purpose of the inventions described herein. For example, the Type 1 interferon may be a beta-interferon (interferon-β 1a or 1b). Examples include as β-interferon (Avonex® (interferon-beta 1a), Rebiff® (by Serono); Biogen, Betaseron® (interferon-beta 1b; Berlex, Schering).

In one embodiment, the beta interferon may be Rebif, Betaseron, or Avonex, or the active ingredients therein. The dosages of each of these currently used are: Avonex-interferon beta-1a, 30 mcg, injected intramuscularly, once a week; Rebif-interferon beta-1a, 44 or 22 mcg, injected subcutaneously, three times per week; Betaseron-interferon beta-1b, 0.25 mg, injected subcutaneously, every other day. The dosage of beta interferon useful in this invention may include lower doses than generally used if a combination with an ERβ ligand is used. For example, Avonex at about 15-29 mcg, Rebif at about 11-21 mcg, or Betaseron at about 0.125-0.24 mg.

Alternatively, a patient may achieve a greater clinical benefit using a dosage at or about the currently approved interferon dose, but adding treatment with ERβ ligand. In one embodiment, the beta interferon may include, for example, a dose of Avonex at about 30 mcg, Rebif at about 22-44 mcg, Betaseron at about 0.25 mg. The current invention may be advantageous at least because many MS patients either do not take low dose interferon treatment (such as Avonex) for fear that it is suboptimally efficacious in controlling their clinical MS, or after having been initially started on low dose interferon, they switch off of it to other treatments for the same reason. This invention would permit patients to start, and stay on, low dose interferon by adding ERβ ligand treatment to it to improve efficacy.

Optionally, the following tertiary agents may be used: glatiramer acetate (Copaxone®; Teva), antineoplastics (such as mitoxantrone; Novatrone® Lederle Labs), human monoclonal antibodies (such as natalizumab; Antegren® Elan Corp. and Biogen Inc.), immonusuppressants (such as mycophenolate mofetil; CellCept® Hoffman-LaRoche Inc.), paclitaxel (Taxol®; Bristol-Meyers Oncology), cyclosporine (such as cyclosporin A), corticosteroids (glucocorticoids, such as prednisone and methyl prednisone), azathioprine, cyclophosphamide, methotrexate, cladribine, 4-aminopyridine and tizanidine

In yet other embodiments, additional agents may be added to the combination at a therapeutically effective amount. Preferably the additional agent may be administered at a lower dose due to the synergistic effect with the combination of the first and second agents. Examples include a glucocorticoid, precursor, analog or glucocorticoid receptor agonist or antagonist. For example, prednisone may be administered, most preferably in the dosage range of about 5-60 milligrams per day. Also, methyl prednisone (Solumedrol) may be administered, most preferably in the dosage range of about 1-2 milligrams per day. Glucocorticoids are currently used to treat relapse episodes in MS patients, and symptomatic RA within this dosage range.

Therapeutically Effective Dosage. A therapeutically effective dose is at least one sufficient to raise the serum concentration above basal levels, and preferably to produce a biological effect on a positive control tissue.

The dosage of each active agent may be selected for an individual patient depending upon the route of administration, severity of disease, age and weight of the patient, other medications the patient is taking and other factors normally considered by the attending physician, when determining the individual regimen and dosage level as the most appropriate for a particular patient.

Dosage Form. The therapeutically effective dose of the active agent(s) included in the dosage form is selected as discussed above. The dosage form may include the active agent(s) in combination with other inert ingredients, including adjutants and pharmaceutically acceptable carriers for the facilitation of dosage to the patient as known to those skilled in the pharmaceutical arts. The dosage form may be any form suitable to cause the agent(s) to enter into the tissues of the patient.

In one embodiment, the dosage form of the agent(s) is an oral preparation (liquid, tablet, capsule, caplet or the like) which when consumed results in elevated levels of the agent(s) in blood serum. The oral preparation may comprise conventional carriers including dilutents, binders, time release agents, lubricants and disinigrants.

Possible oral administration forms are all the forms known from the prior art such as, tablets, dragees, pills or capsules, which are produced using conventional adjuvants and carrier substances. In the case of oral administration it has provided appropriate to place the daily units of agent(s), in a spatially separated and individually removable manner in a packaging unit, so that it is easy to check whether the typically daily taken, oral administration form has in fact been taken as it is important to ensure that there are no taking-free days.

In other embodiments of the invention, the dosage form may be provided in a topical preparation (lotion, crème, ointment, patch or the like) for transdermal application. Alternatively, the dosage form may be provided in a suppository or the like for intravaginal or transrectal application. Alternatively, the agents may be provided in a form for injection or for implantation.

That the agents could be delivered via these dosage forms is advantageous in that currently available therapies, for MS for example, are all injectables which are inconvenient for the user and lead to decreased patient compliance with the treatment. Non-injectable dosage forms are further advantageous over current injectable treatments which often cause side effects in patients including flu-like symptoms (particularly, β interferon) and injection site reactions which may lead to lipotrophy (particularly, glatiramer acetate copolymer-1).

However, in additional embodiment, the dosage form may also allow for preparations to be applied subcutaneously, intravenously, intramuscularly or via the respiratory system.

### EXAMPLE 1

Material and Methods: Animals: B6.Cg-Tg (Thy1-YFP) 16Jrs/J (Thy1-YFP) mice 8-10 weeks old were purchased from the Jackson Laboratory (Bar Harbor, Me.). Animals were maintained under environmentally controlled conditions in a 12 hour light/dark cycle with access to food and water ad libitum. All procedures involving animals were carried out in accordance to the NIH guidelines for the care and use of laboratory animals and approved by the UCLA Chancellor's Animal Research Committee and Division of Laboratory Animals Medicine.

Reagents: The ERβ ligand Diarylproprionitrile (DPN) was purchased from Tocris Biosciences (Ellisville, Mo.) and dissolved with molecular grade ethanol purchased from EM Sciences (Hatfield, Pa.). Miglylol 812N liquid oil was Sasol North America (Houston, Tex.). Recombinant mouse Interferon-beta (IFNβ) was purchased from PBL InterferonSource (Piscataway, N.J.). All reagents were prepared and stored according to manufacturer's instructions.

EAE induction and treatments: Animals were injected subcutaneously with Myelin Oligodendrocyte Glycoprotein (MOG), amino acids 35-55 (200 μg/animal, American Peptides), emulsified in complete Freund's adjuvant (CFA) and supplemented with Mycobacterium Tuberculosis H37ra (200 μg/animal, Difco Laboratories), over four draining inguinal and axillary lymph node sites in a volume of 0.1 ml/mouse. Seven days prior to immunization, animals received treatment that continued to the endpoint of the experiment with DPN (8 mg/kg/day, s.c. injections) dissolved in 10% molecular-grade ethanol and diluted with 90% Miglylol 812N liquid oil, rmIFNβ (20 KU, i.p. injections) diluted with injection grade PBS and 0.1% FBS carrier protein, vehicle consisting of 1:9 molecular grade ethanol/Miglylol 812N, or a combination of DPN and IFNβ. Animals were monitored daily for EAE signs based on a standard EAE 0-5 scale scoring system: 0-healthy, 1-complete loss of tail tonicity, 2-loss of righting reflex, 3-partial paralysis, 4-complete paralysis of one or both hind limbs, and 5-moribund.

Histological preparation: Mice were deeply anesthetized in isoflurane and perfused transcardially with ice-cold 1× PBS for 20-30 minutes, followed by 10% formalin. Spinal cords were dissected and submerged in 10% formalin overnight at 4° C., followed by 30% sucrose in PBS for 24 hours. Spinal cords were cut in thirds and embedded in 75% gelatin/15% sucrose solution. 40 μm thick free-floating spinal cord cross-sections were obtained with a microtome cryostat (model HM505E) at −20° C. Tissues were collected serially and stored in 1× PBS with 1% sodium azide in 4° C. until immunohistochemistry.

Immunohistochemistry: 40 μm thick free-floating sections were thoroughly washed with 1× PBS to dilute residual sodium azide. In the case of anti-MBP labeling, tissue sections undergo an additional 2 hour incubation with 5% glacial acetic acid in 100-proof ethanol at room temperature (RT), followed by 30 minutes incubation in 3% hydrogen peroxide in PBS. All tissue sections were permeabilized with 0.3% Triton X-100 in 1× PBS and 2% normal goat serum (NGS) for 30 minutes RT, and blocked with 10% NGS in 1× PBS, except in the case of MBP labeling, which was blocked with 10% normal sheep serum (NSS), for 2 hours or overnight at 4° C. The following primary antibodies (Abs) were used: anti-sheep MBP (1:1000), anti-CD45 (1:500), anti-CD3 (1:500), anti-Mac3 (1:500) (Chemicon), and anti-neurofilament-NF200 (1:750, Sigma). Tissues labeled with anti-sheep MBP continue with second Ab labeling step consisting of 1 hour incubation with biotinylated anti-sheep IgG Ab (1:1000, Vector Labs), followed by 1½ hour incubation with strepavidin Ab conjugated to Alexa 647 fluorochrome (Chemicon). All other tissues followed with second Abs conjugated to TRITC (1:1000) or Cy5 (1:750) (Vector labs and Chemicon) for 1½ hours. To assess the number of cells, a nuclear stain DAPI (2 ng/ml, Molecular Probes) was added 10 minutes prior to final washes after secondary Ab incubation. Sections were mounted on slides, allowed to semi-dry, and cover slipped in fluoromount G (Fisher Scientific).

Microscopy: Stained sections were examined and photographed using a confocal microscope (Leica TCS-SP, Mannheim, Germany) or a fluorescence microscope (BX51WI; Olympus, Tokyo, Japan) equipped with Plan Fluor objectives connected to a camera (DP70, Olympus). Digital images were collected and analyzed using Leica confocal and DP70 camera software. Images were assembled using Adobe Photoshop (Adobe Systems, San Jose, Calif.).

Quantification: To quantify immunohistochemical staining results, three spinal cord cross-sections at the T1-T5 level from each mouse (n=3) were captured under microscope at 10× magnification for YFP/CD45 labeled sections, or 40× magnification for YFP/MBP labeled sections using the DP70 Image software and a DP70 camera (both from Olympus). All images in each experimental set were captured under the same light intensity and exposure limits. Analysis was performed on images using ImageJ Software v1.30, downloaded from the NIH website: http://rsb.info.nih.gov/ii. Inflammatory infiltrates were quantified by measuring the intensity of CD45 staining in the lateral funiculus in captured 10× images. Axons were identified by YFP expression in the lateral funiculus in captured 40× images and quantified with the measure function in the ImageJ software.

Splenocyte culture: Splenocytes were cultured in 24-well plates at the concentration of 4×10⁶ cells/ml of complete RPMI medium containing 5% heat-inactivated fetal calf serum (FCS), 1 mM sodium pyruvate, L-glutamine, 2ME, NEAA, Pen-strep, and 25 mM Hepes Buffer. Cells were stimulated with 25 μg/ml MOG, amino acids 35-55, and 20 ng/ml IL-12 (BD Biosciences) for 72 hours at 37° C., 5% CO₂. After 72 hours of culture, supernatants were collected and centrifuged to eliminate cellular debris prior to flash freezing in isopropanol and dry ice and stored in −80° C. until ready for analysis. Cytokine analyses were performed by Searchlight Array (Thermo Fisher Scientific).

Flow cytometry: Splenocytes were collected on a 96 v-shaped plate (Titertek Co.) for flow cytometric analysis. Single cell suspensions in FACs buffer (2% FCS in PBS) were incubated with anti-CD16/32 at 1:100 dilution for 20 minutes at 4° C. to block Fc receptors, centrifuged, and resuspended in FACs buffer with the following Abs added at 1:100 dilution for 30 minutes at 4° C.: anti-CD11b, anti-CD11c, anti-CD19, anti-CD4, Rat-IgG1, -IgG2a, and -IgG2b isotype controls (Biolegend). Cells were subsequently washed twice in FACs buffer, acquired on FACSCalibur (BD Biosciences) and analyzed using Flowjo Software (Treestar).

Statistical analysis: EAE severity significance was determined by one-way Repeated Measure Analysis of Variance (ANOVA). Statistical analysis of the data is represented as Mean±Standard Error of pooled EAE scores. In the case where the scatter plot of immunohistochemical or flow cytometry data satisfied assumptions of normal distribution and equal variances among all groups, the data were analyzed by bootstrap one-way ANOVA and student's t-test, respectively. For these analyses, the mean or median was used as the comparator, and F-stat equation was modified such that absolute values replaced the squaring of values. For bootstrap one-way ANOVA, post-hoc analysis was performed on F-stat values at 95% confidence interval.

Results

Combination Treatment with IFNβ and ERβ Ligand Significantly Reduced EAE Disease Severity.

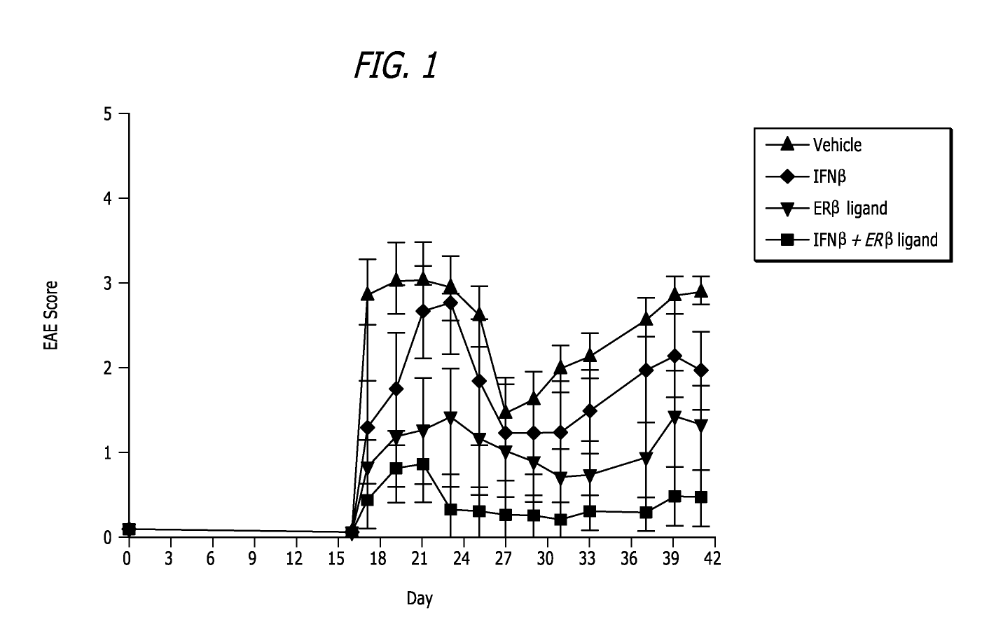

To pursue possible additive effects between two therapeutic agents in EAE, we first examined various doses of IFNβ treatment in EAE. It had previously been shown that 10 KU of IFNβ was effective in reducing mean clinical disease scores in EAE in the SJL/J strain, therefore we included this dose as well as three other doses: 5 KU, 15 KU, and 20 KU. The two lower doses (5 KU and 10 KU) failed to reduce EAE scores in C57BL/6 mice, but the two higher doses (15 KU and 20 KU) worked comparably in reducing mean clinical scores as compared to vehicle treated. Notably, the highest dose of 20 KU resulted in only mild reductions in EAE scores, consistent with observations by others. Thus, 20 KU was chosen for subsequent experiments using combination treatment. The dose of the ERβ ligand which could reduce EAE scores was previously established in our lab. We then determined whether combination treatment using an ERβ ligand with IFNβ might be additive in reducing EAE clinical scores. As shown in FIG. 1, there was a trend for IFNβ treatment alone to reduce the severity of EAE when compared to vehicle treated groups, but this did not reach significance. In contrast, combination treatment using IFNβ with the ERβ ligand resulted in lower mean clinical scores compared with vehicle or IFNβ treatment alone. Indeed, mice in the combination treatment group showed near complete clinical recovery from day 23 to endpoint at day 40 (p<0.001, FIG. 1). The clinical benefit of combination treatment was sustained, as demonstrated in another experiment in which animals were treated to a later time point, day 70 (p=0.001, not shown). These results show that combining ERβ ligand treatment with IFNβ treatment is additive with respect to its effect on clinical EAE.

As shown in FIG. 1, combination treatment using IFNβ with ERβ ligand was additive in reducing EAE. Mean clinical scores±SD of EAE mice treated with vehicle (black), IFNβ (red), ERβ ligand (orange), or the combination (green). In mice treated with IFNβ alone, there was a trend for reduced disease as compared to vehicle treated, but this did not reach significance. In contrast, combination treatment of IFNβ and the ERβ ligand significantly reduced EAE from the onset of disease to the endpoint of the experiment at day 40 (p<0.0001).

Combination Treatment with IFNβ and ERβ Ligand Preserved Axon Densities in Spinal Cords of EAE Mice.

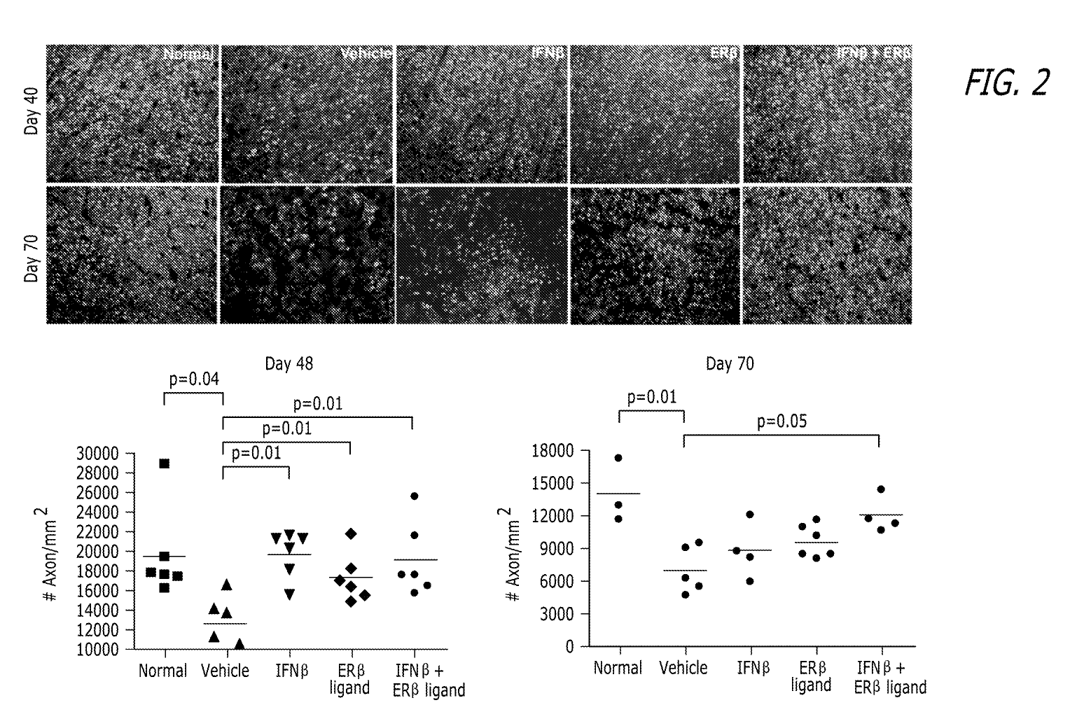

Axonal loss has been proposed as a neuropathologic substrate for clinical disease severity in EAR We had previously shown that ERβ ligand treatment preserved axon densities in spinal cords of EAE mice. To determine the effect of combination treatment on axonal loss, we examined thoracic spinal cords of treated EAE mice. Since the mice used in our experiments were transgenic for yellow fluorescent protein (YFP) which is driven by the neuronal-specific thy1 promoter, YFP served as an axonal marker. Indeed, staining in spinal cord sections with the neuronal marker NF200 completely co-localized with YFP expression (not shown). Hence, spinal cord cross sections were directly examined for YFP⁺ axons. As shown in FIG. 2, combination treatment preserved axonal densities in the spinal cord during EAE as compared to vehicle treated (p=0.01, one-way ANOVA), at day 40 of EAE. Also, as previously reported, treatment with ERβ ligand alone preserved axon densities. Surprisingly, IFNβ treatment alone preserved axonal densities despite the lack of a significant effect of IFNβ treatment on clinical EAE severity (FIG. 2, p=0.01). It was possible that the anti-inflammatory properties of IFNβ merely delayed, but did not prevent axonal loss. Thus, we next examined spinal cord sections in another set of mice which were sacrificed at a later time point, day 70. Similar to the results at day 40, combination treatment continued to preserve axonal densities up to day 70 as compared to vehicle treatment (FIG. 2, p=0.05). However, at this later time, neither IFNβ nor ERβ ligand treatment alone significantly prevented axonal loss. These results show that ERβ ligand treatment in combination with IFNβ treatment is additive with respect to preserving axon densities in spinal cords of mice at relatively late stages of EAE.

As shown in FIG. 2, combination treatment using IFNβ with ERβ ligand preserved axonal densities in the spinal cord of EAE mice. 40× images of the lateral funiculus of the thoracic spinal cord of day 40 and day 70 EAE mice were stained for myelin with MBP. Yellow-fluorescent-protein (YFP) expression identified axons. At day 40, IFNβ treatment alone, ERβ ligand treatment alone, and combination treatment significantly preserved axonal densities compared to vehicle treated (p=0.01). By day 70, only combination treatment continued to significantly preserve axonal densities compared to vehicle treated (p=0.05).

Combination Treatment with IFNβ and ERβ Ligand is Additive in Reducing Infiltration of T Cells and Macrophages into the CNS of EAE Mice.

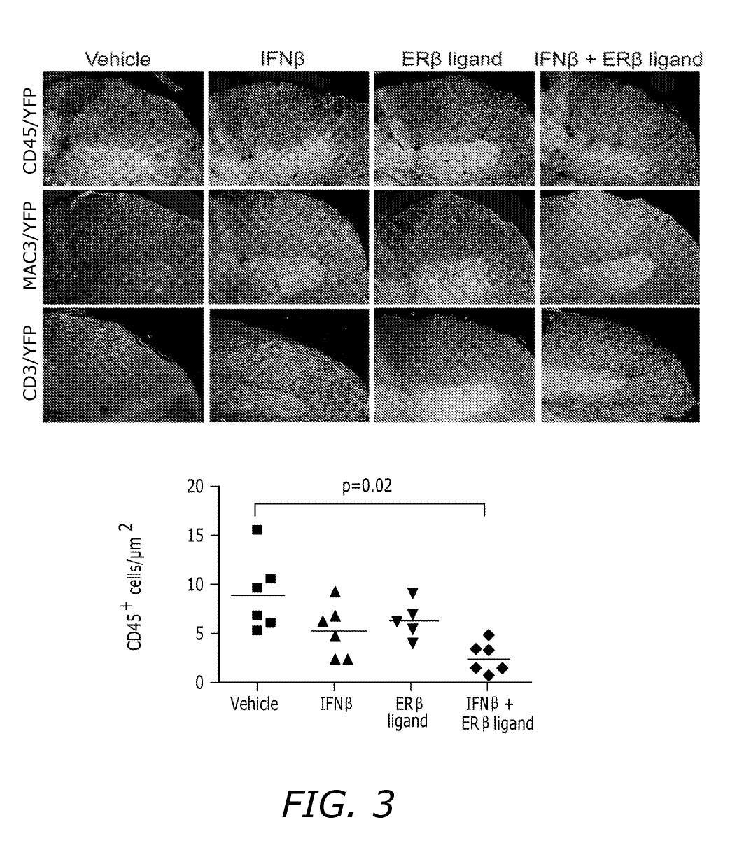

One of the primary actions of IFNβ is to reduce inflammation in the CNS. To determine whether the addition of ERβ ligand treatment influenced this effect of IFNβ, we assessed the degree of inflammation in spinal cords of EAE mice treated with vehicle, IFNβ alone, ERβ ligand alone, or the combination. At the endpoint of disease (day 40), thoracic spinal cord sections were examined for CD45⁺ cells, a pan-immune cell marker, by immunohistochemistry. While a trend existed, IFNβ treatment alone did not significantly reduce the infiltration of CD45⁺ cells into the CNS of EAE mice, as compared to vehicle treated (FIG. 3). Similar to our previous experiments in active EAE [18], ERβ ligand treatment alone here in adoptive EAE did not decrease inflammation as compared to vehicle treatment. In contrast, combination treatment with IFNβ and ERβ ligand significantly reduced CD45⁺ staining in the CNS of EAE mice (p=0.02, one-way ANOVA).

To determine which immune cell types were affected by treatment, these thoracic spinal cord sections were also examined for CD3⁺ T cells and Mac3⁺ macrophages. Combination treatment reduced staining for both T cells and macrophages in the CNS (FIG. 3).

As shown in FIG. 3, combination treatment using IFNβ with ERβ ligand reduced inflammatory cell infiltration in the spinal cord of EAE mice. 10× images of the lateral funiculus of the thoracic spinal cord of day 40 EAE mice were stained for the pan-immune cell marker CD45 (top), Mac3 (middle), and CD3 (bottom). YFP expression identified neurons and axons. Vehicle treated mice exhibited high levels of inflammation in the CNS. Mac3 and CD3 staining revealed that inflammatory infiltrates consisted of macrophages and T cells, respectively. There was a trend for IFNβ treatment alone and ERβ ligand treatment alone to decrease CD45 staining as compared to vehicle treated, but this did not reach significance. In contrast, combination treatment using both IFNβ and ERβ ligand significantly reduced CD45 staining as compared to vehicle treated (p=0.02).

ERβ Ligand Antagonizes IFNβ Treatment Effects on Th1 and Th2 Cytokine Levels.

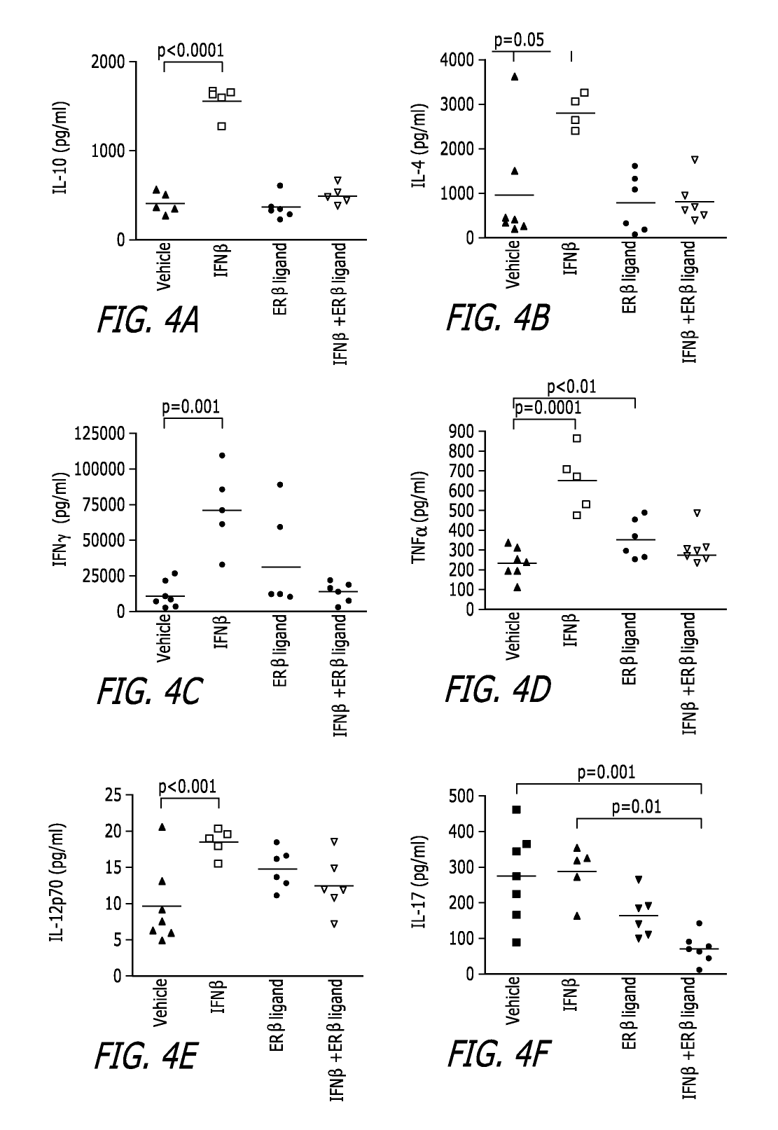

It had previously been shown that IFNβ treatment alone affected cytokine production of peripheral immune responses, while ERβ ligand treatment alone did not. Thus, we next assessed cytokine levels (IL-10, IL-4, TNFα, IFNγ, IL-12p70, and TGFβ) upon ex vivo stimulation of splenocytes with autoantigen at day 40 of EAE. Treatment with IFNβ alone significantly increased levels of the Th2 cytokines IL-10 (FIG. 4A, p<0.0001) and IL-4 (FIG. 4B, p=0.001). Interestingly, the Th1 cytokines IFNγ (FIG. 4C, p<0.0001), TNFα (FIG. 4D, p=0.0001), and IL-12p70 (FIG. 4E, p=0.001) were also increased with IFNβ treatment (p-values by one-way ANOVA, FIG. 4). Consistent with previous literature, ERβ ligand treatment did not significantly alter cytokine levels as compared to vehicle. Surprisingly, the addition of ERβ ligand treatment to IFNβ in the combination treatment arm resulted in abrogation of the immunostimulatory effects of IFNβ treatment on cytokines. We repeated these analyses in another experiment in which the animals were sacrificed at day 70 and achieved similar results (data not shown). Thus, while IFNβ and ERβ ligand treatments were additive with respect to clinical and neuropathologic outcomes, ERβ ligand treatment in combination with IFNβ abrogated IFNβ mediated effects on Th1 and Th2 cytokines by peripheral immune cells in both the early and later time points of disease.

As shown in FIG. 4, treatment with ERβ ligand in combination with IFNβ antagonized the stimulatory effect of IFNβ on Th1 and Th2 cytokines, while it reduced Th17 cytokine levels. Th1, Th2 and Th17 cytokine levels measured from supernatants of splenocytes stimulated with MOG 35-55 revealed that IFNβ treatment alone increased IL-10 (4A, p<0.0001), IL-4 (4B, p=0.001), IFNγ (4C, p<0.0001), TNFα (4D, p=0.0001), and IL-12p70 (4E, p=0.00), as compared to vehicle treatment. IFNβ treatment alone did not affect levels of IL-17 (4F). ERβ ligand treatment alone had no effect on Th1 and Th2 cytokine levels, but when combined with IFNβ, it negated the changes seen with IFNβ treatment alone. ERβ ligand treatment alone tended to decrease levels of the Th17 cytokine IL-17, but this did not reach significance. However, when ERβ ligand was combined with IFNβ, it additively reduced the levels of IL-17 as compared to IFNβ treatment alone (4F, p=0.01) and vehicle treatment (4F, p=0.001).

Combination Treatment with IFNβ and ERβ ligand are additive in reducing IL-17 levels.

Th17 cells have been shown to play an important role in EAE, particularly during the later, more chronic phase of disease. Since we had observed significant axonal sparing relatively late in disease with combination treatment, we next determined the levels of Th17 cytokine production during treatment with IFNβ alone, ERβ ligand alone, or the combination. Supernatants from autoantigen stimulated splenocytes from EAE mice were analyzed for IL-17 and IL-23. Interestingly, IFNβ treatment alone did not affect the levels of IL-17, whereas ERβ ligand treatment alone showed a trend towards decreased IL-17 production, but this did not reach significance (FIG. 4F). In contrast, combination treatment with IFNβ and ERβ ligand significantly reduced levels of IL-17 as compared to vehicle treatment (FIG. 4F, p=0.02, one-way ANOVA). Since IL-23 is a key cytokine to maintaining Th17 activity, we also examined levels of IL-23 and found that they were no different between any treatment groups (not shown). Thus, in contrast to antagonistic effects of combination treatment on Th1 and Th2 cytokines, combination treatment significantly reduced IL-17 levels from autoantigen stimulated splenocytes.

ERβ Ligand treatment Antagonizes IFNβ Effects on MMP-9.

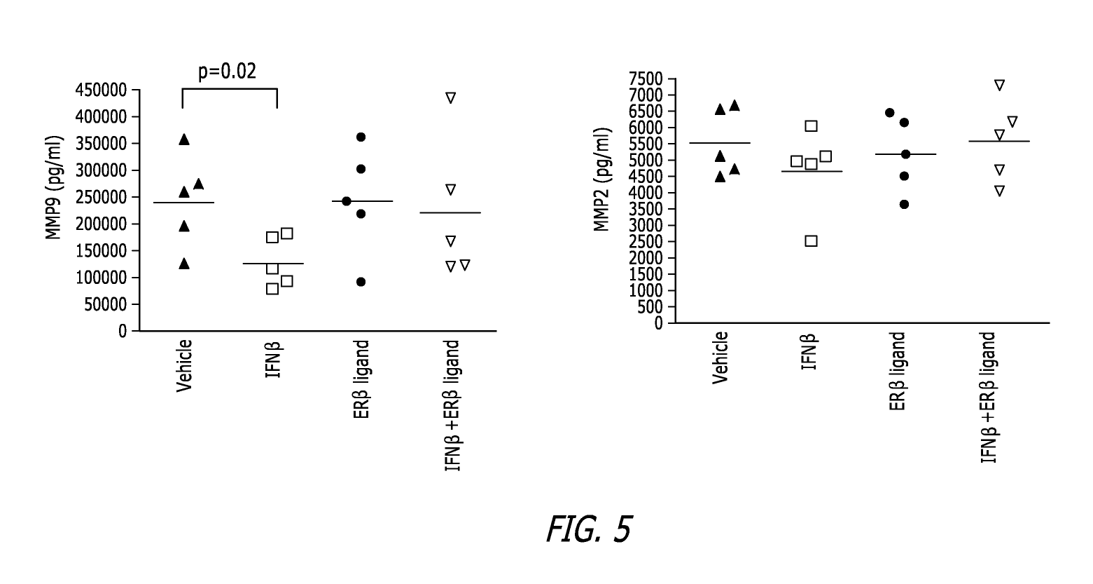

In light of the additive effect of combination treatment on CNS inflammation (FIG. 3), we next focused on molecules involved in immune cell trafficking to the CNS. In MS and EAE, MMP-9 and MMP-2 can be involved in mediating inflammation in the CNS. We therefore assessed the effect of IFNβ, ERβ ligand, or combination treatment on MMP-9 and MMP-2 expression by autoantigen stimulated splenocytes from mice with EAE. Consistent with work in MS, treatment with IFNβ alone significantly reduced MMP-9 in EAE (p=0.002), while MMP-2 was unchanged, as compared to vehicle treated (FIG. 5). ERβ ligand treatment alone had no effect on MMP expression. Surprisingly, the addition of ERβ ligand treatment to IFNβ treatment antagonized the IFNβ-mediated decrease in MMP-9. Therefore, with respect to both Th1 and Th2 cytokine production and MMP-9 expression, ERβ ligand treatment in combination with IFNβ abrogated the immunomodulatory effects of IFNβ treatment.

As shown in FIG. 5, treatment with ERβ ligand in combination with IFNβ antagonized the effect of IFNβ on reducing MMP-9. IFNβ treatment alone significantly decreased MMP-9 levels in supernatants as compared to vehicle treated, while ERβ ligand treatment alone did not affect production of MMPs (p=0.02). The addition of ERβ ligand treatment to IFNβ during combination treatment abrogated the effect of IFNβ on MMP-9. There were no differences in MMP-2 between any of the treatment groups.

Combination Treatment with IFNβ and ERβ Ligand are Additive in Reducing VLA-4 Expression on CD4⁺ T Cells in EAE.

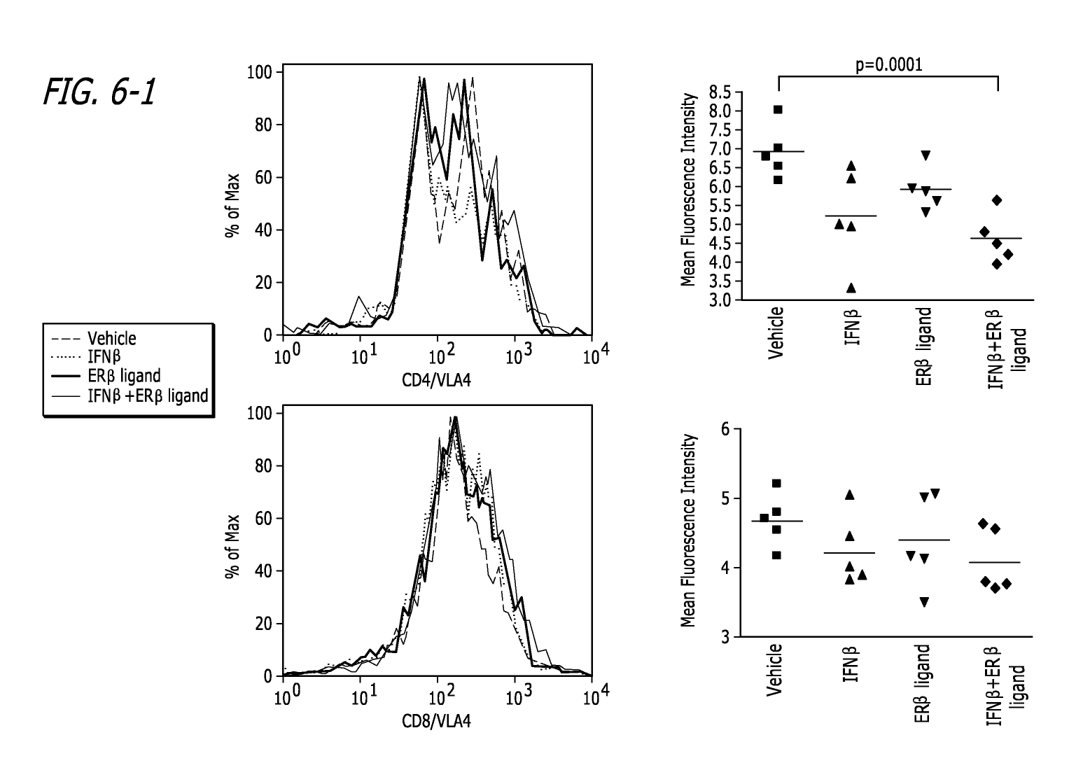

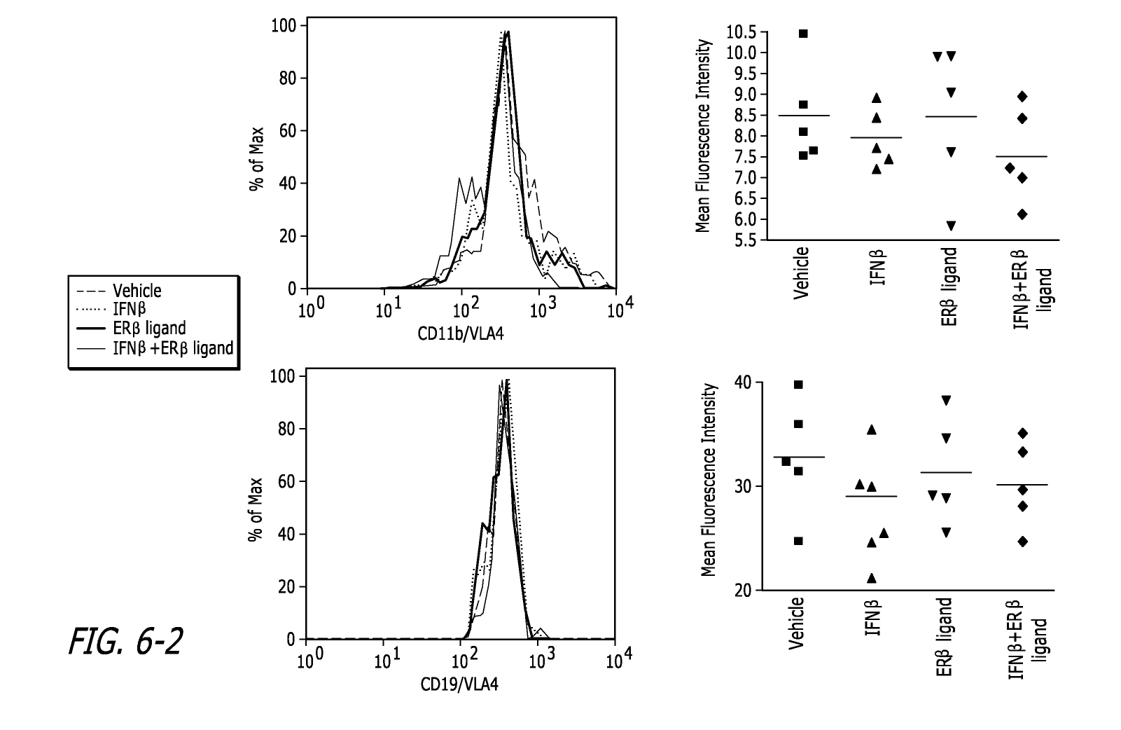

To explore other potential transmigratory factors underlying additive clinical and neuropathologic effects, we next focused on a critical cell adhesion molecule. VLA-4 (CD49d) is known to play an important role in immune cell trafficking in both MS and EAE. Splenocytes from EAE mice treated with either IFNβ, ERβ ligand or the combination were stimulated ex vivo with autoantigen and analyzed for expression of VLA-4 on T cells, B cells, and macrophages. There was a trend towards decreased VLA-4 expression on CD4⁺ T cells with IFNβ treatment alone compared to vehicle treated, but this did not reach significance, and there was no effect of ERβ ligand treatment alone (FIG. 6). In contrast, the expression of VLA-4 was significantly lower on CD4⁺ T cells of EAE mice treated with the combination (p=0.0001). There were no differences in VLA-4 expression on CD8⁺, CD19⁺, or CD11b⁺cells between any treatment groups. These results demonstrated that combining ERβ ligand treatment with IFNβ treatment was additive with respect to decreasing VLA-4 expression on CD4⁺ T cells in EAE, consistent with the additive effect of these two treatments on reducing inflammation in the CNS (FIG. 3).

As Shown in FIG. 6, Treatment with ERβ Ligand in Combination with IFNβ Reduced VLA-4 Expression on CD4⁺ T cells of EAE mice. Representative histograms of the level of VLA-4 expression on gated CD4 and CD8 (T cells), CD19 (B cells), and CD11b (macrophages and monocytes). There was a trend for IFNβ treatment alone and ERβ ligand treatment alone to decrease VLA-4 expression on CD4⁺ T cells, but this did not reach significance. In contrast, combination treatment using both IFNβ and ERβ ligand significantly reduced VLA-4 expression (p=0.0001, blue), as compared to vehicle (red) treated mice. No differences in VLA-4 expression were observed on CD8, CD19 and CD11b cells between any treatment groups.

### EXAMPLE 2

Methods: Animals: Breeding pairs of PLP_EGFP mice on the C57BL/6J background were a kind gift from Dr. Wendy Macklin (University of Colorado, Denver). The generation, characterization and genotyping of PLP_EGFP transgenic mice have been previously reported. Mice were bred in house at the University of California, Los Angeles animal facility. All procedures were conducted in accordance with the National Institutes of Health (NIH) and were approved by the Animal Care and Use Committee of the Institutional Guide for the Care and Use of Laboratory Animals at UCLA.

Reagents: Diarylpropionitrile (DPN) was purchased from Tocris Bioscience (Ellisville, Mo.). Miglyol 812 N liquid oil was obtained from Sasol North America (Houston, Tex.). MOG peptide, amino acids 35-55, was synthesized to >98% purity by Mimotopes (Clayton, Victoria, Australia).

Hormone Manipulations: Female mice (6 weeks old) were ovariectomized two weeks prior to induction of EAE. Ovariectomized mice were treated with subcutaneous injections of DPN at 8 mg/kg/day or vehicle (10% ethanol and 90% Migylol) every other day beginning 7 days before EAE induction and throughout the entire disease duration. The DPN dose was chosen based on uterine weight measurements for biological response and on previous EAE experiments using this compound (Tiwari-Woodruff S, et al., “Differential neuroprotective and antiinflammatory effects of estrogen receptor (ER)alpha and ERbeta ligand treatment.” Proc Natl Acad Sci USA 2007; 104: 14813-8.).

Results: Treatment Reduces Clinical Disease Severity Scores in EAE. To visualize and characterize ERβ ligand treatment effects on demyelination and axon degeneration, active EAE was induced in proteolipid protein-enhanced green fluorescent protein (PLP_EGFP) transgenic C57Bl/6. To obtain a steady level of ERβ ligand diarylpropionitrile (DPN) dose of 8 mg/kg/day, ERβ ligand or vehicle treatment was administered in ovariectomized mice every other day starting one week prior to active EAE induction. Ovariectomized mice showed similar EAE disease time course and clinical scores as intact animals (Supplementary FIG. 1A). ERβ ligand treatment during EAE had no significant effect early on, that is prior to day 20, but thereafter demonstrated a significant protective effect throughout the later stages of disease, p<0.001 (FIG. 1A).

As shown in FIG. 1, treatment with ERβ ligand significantly improves disease in late chronic EAE.

(A) Ovariectomized PLP_EGFP C57BL/6 female mice were given subcutaneous injections of diarylpropionitrile, an estrogen receptor beta (ERβ) ligand, during active EAE and scored using the standard EAE grading scale. ERβ ligand treated mice, as compared to vehicle treated mice, were not significantly different early in disease (up to day 20 after disease induction), but then became significantly improved later during EAE, (starting at day 22-25 after disease induction, p<0.001, ANOVA Friedman test). Normal mice did not show any disease and their clinical scores remained zero through out the experiment. Number of mice in each group were normal, n=6; EAE+vehicle, n=6; EAE+ERβ ligand, n=8. Data are representative of experiments repeated three times.

(B) Brain slices for immunohistochemistry corresponded approximately to plates 29-48 in the atlas of Franklin and Paxinos. (CC: corpus callosum; Hip: hippocampus; S1: somatosensory cortex; M: motor cortex).

(C) Representative PLP_EGFP expressing (green) and DAPI nuclei (blue) stained CC sections (10× magnification) from normal (healthy control), vehicle treated EAE, and ERβ ligand-treated EAE mice all sacrificed at day 36 (late) post-disease induction. Compared to normal controls, the CC of vehicle-treated EAE and ERβ ligand-treated EAE had an increase in the total number of infiltrating cells (represented by DAPI⁺ cells) after induction of EAE. This was accompanied by a reduction in PLP_EGFP⁺ cells, as well as PLP_EGFP white matter intensity (white arrows). Scale bar is 100 μm.

As shown in FIG. 1B, EAE clinical scores were similar in intact and ovariectomized mice.

(A) Active EAE was induced with MOG peptide in age matched intact and ovariectomized PLP_EGFP C57LL/6 female mice and scored using the standard EAE grading scale. There was no significant difference in early or late disease. Normal intact and ovariectomized mice did not show any disease and their clinical scores remained zero through out the experiment. Number of mice in each group were intact normal, n=4; intact EAE, n=6; ovariectomized normal, n=6, gonadectomized EAE, n=6.

(B-C) Representative PLP_EGFP expressing (green), MBP (red) and DAPI nuclei (blue) stained posterior funniculus of thoracic spinal cord and brain callosal sections (10× magnification) from intact normal and intact EAE mice all sacrificed at day 36 (late) post-disease induction. Compared to intact normal mice, the dorsal column (DC) and CC of EAE mice had an increase in the total number of infiltrating cells (represented by DAPI⁺ cells) after induction of EAE. This was accompanied by a reduction in PLP_EGFP⁺ cells, as well as PLP_EGFP white matter and MBP immunostaining intensity (white arrows). Scale bar is 100 μm.

Inflammation and Reactive Astrocytosis in the Corpus Callosum of Mice with EAE

The corpus callosum (CC) that connects both cerebral hemispheres is by far the largest fiber tract in the brain and is preferentially involved in MS. It is widely believed that rodent EAE rarely affects the brain and is mostly limited to pathology of the spinal cord. Contrary to this belief, we have observed extensive callosal and cortical pathology, in addition to spinal cord pathology of both intact and ovariectomized EAE mice (Supplementary FIG. 1-2). PLP_EGFP fluorescing green cells and myelin in CC (delineated region in FIG. 1B) stained with nuclear stain DAPI (blue) allowed us to easily visualize inflammatory and demyelinating lesions in the callosal white matter (arrows, FIG. 1C) and thoracic spinal cord (Supplementary FIGS. 1B and 2). Demyelinating lesions in vehicle treated EAE lacked normal expression of PLP_EGFP OLs and myelin tracts, whereas, ERβ ligand treated EAE CC and spinal cord indicated increased numbers of PLP_EGFP OLs and myelinated tracts along with pockets of infiltrating DAPI nuclei (arrows, FIG. 1C, Supplementary FIG. 2A).

Similar to inflammatory cells seen in the spinal cord from EAE mice (Supplementary FIG. 2), the CC of early and late vehicle-treated EAE mice had many CD45⁺ cells with activated microglia morphology, along with Mac3⁺ macrophage and CD3⁺ T lymphocytes surrounding lesions and vessels (FIG. 2A showing only the late time point). In addition there was a marked increase in the immunoreactivity intensity of GFAP⁺ astrocytes in vehicle-treated EAE animals (FIG. 2A). ERβ ligand treatment did not reduce inflammatory cells or reactive astrocyte levels (FIG. 2A). Quantitative analysis of CD45⁺, Mac3⁺, CD3⁺ and GFAP⁺ cells showed a significant increase in the CC of vehicle-treated EAE compared to normal that was also observed in EAE mice treated with ERβ ligand (FIG. 2B).

As shown in FIG. 2, treatment with ERβ ligand did not reduce inflammation or reactive astrocytosis in the CC of mice with EAE.

(A) Consecutive CC sections were also immunostained with antibodies against the common leukocyte antigen-CD45 (red—at 10× magnification), the macrophage-Mac3 (red—at 40× magnification), the T cell-CD3 (red—at 40× magnification) or the astrocyte marker glial fibrillary astrocytic protein (GFAP, red—at 10× magnification). Shown are images from normal control, vehicle-treated EAE, and ERβ ligand-treated EAE CC at day 36 after disease induction. Vehicle-treated EAE and ERβ ligand-treated CC had large areas of CD45⁺, Mac3⁺ and CD3⁺ cells in the CC as compared to the normal control, as well as large areas of hypertrophic-reactive GFAP⁺ astrocytes.

(B) Quantification of number of CD45+, Mac3+, and CD3+ cells and the relative fluorescence intensity of GFAP immunostaining demonstrated an increase in both vehicle treated EAE mice and ERβ ligand treated EAE as compared to normal mice. Statistically significant compared with normal (**p<0.001 ANOVAs; Bonferroni's multiple comparison post-test; n=8-10 mice in each treatment group).

As shown in Supplementary FIG. 2, EAE induced spinal cord inflammation and axon degeneration is similar in intact and ovariectomized mice.

(A-D) Shown here are representative thoracic spinal cord brain sections from age-matched intact (normal and day 36 EAE) and ovariectomized (normal, day 36 EAE+vehicle, and day 36 EAE+ERβ ligand) animals (A and B respectively). Infiltrating CD45⁺ microglia (red) are imaged at 10× and dashed box inset at 40× are seen in EAE and EAE+ERβ ligand treated dorsal column. Second panel shows NF200⁺ (red) axons imaged at 40× in the dorsal column. Compared to intact normal mice, the dorsal column of EAE mice and EAE+ERβ ligand-treated had an increase in the total number of infiltrating CD45⁺ after induction of EAE (C). Axon damage assessed by counting NF200⁺ axons showed significant decreases in EAE animals but not in normal or ERβ ligand treated mice (D). Scale bar is 100 μm. (*p<0.05; **p<0.001, ANOVAs; Bonferroni's multiple comparison post-test; n=4)

ERβ Ligand Treatment During EAE Maintains a Robust OL Population

To address the possible cause of the improved state of PLP_EGFP cells and myelin tracts in ERβ ligand treated EAE mice, the cells of OL lineage were quantified in the delineated CC. The PLP_EGFP fluorescent OL population in the CC of vehicle-treated EAE mice showed patches of decreased intensity, retracted cell processes and smaller cell bodies (FIG. 2A, 3Ai, ii) compared to normal mice. ERβ ligand treated EAE mice had increased numbers of highly processed cells with normal sized cell bodies (FIG. 2A, 3Ai, ii). Quantification of PLP_EGFP⁺ cells indicated a significant decrease in the CC of vehicle-treated EAE mice compared to normal controls. In contrast PLP_EGFP⁺ cell numbers in ERβ ligand treated EAE mice were not decreased (FIG. 3B).

The PLP_EGFP cell populations in the CC are a mixture of OL progenitors (OLP) and mature OLs. Therefore, we quantified OLPs by immunostaining with olig2 or platelet derived growth factor receptor-alpha (PDGFR-α) antibody and did not observe significant differences between vehicle and ERβ ligand treated groups (FIG. 3A-B). The mature OL population was quantified by counting cells that express the mature OL marker, glutathione-S transferase-pi (GST-pi). Compared to normal mice, the CC of vehicle treated EAE mice had ˜25% less GST-pi⁺ cells. In contrast, ERβ ligand treated EAE mice had significantly more GST-pi⁺ cells than vehicle treated mice and were similar to normal OL numbers (FIG. 3A, B).

As shown in FIG. 3, Treatment with an ERβ ligand preserved mature myelinating OLs in CC of mice with EAE.

(A) Shown are representative CC sections with PLP_EGFP⁺ cells (green) from normal, vehicle-treated, and ERβ ligand-treated EAE mice all sacrificed at day 36 (late) post-disease induction (i-10× magnification, ii-40× magnification of the white dashed boxes in panel i). Compared to the CC of vehicle-treated EAE mice, the number of PLP_EGFP⁺ cells was significantly increased in ERβ ligand treated EAE. PLP_EGFP⁺+DAPI⁺ cells had more processes and were in clusters of >3 cells in ERβ ligand treated CC compared to cells that were smaller and with fewer processes in vehicle treated EAE CC. Consecutive brain slices were also immunolabeled with olig2 (red) +DAPI or GST-pi (red) +DAPI (iii, iv-10× magnification, inset 40× magnification). Olig2⁺ cell density under all three conditions showed no obvious difference (iii). The GST-pi⁺ mature OL cell population decreased in vehicle treated EAE compared to normal control CC. There is a dramatic increase in the GST-pi cell population in ERβ ligand treated EAE CC. Platelet growth factor receptor-α (PDGFRα-red) is a specific marker for OLPs. Similar to olig2, PDGFRα⁺ OLPs did not show a significant difference between normal, vehicle-treated EAE and ERβ ligand-treated EAE groups (iv).

(B) Quantification of the number of PLP_EGFP⁺, olig2⁺ and GST-pi⁺ cells per 400 μm² indicated a significant decrease in the number of PLP_EGFP⁺ cells, no change in olig2⁺ cells and a significant decrease in GST-pi⁺ cells in vehicle-treated EAE mice compared to normal controls. ERβ ligand treatment caused a significant increase in PLP_EGFP⁺ cells, no change in olig2⁺ and a significant increase in GST-pi⁺ cells compared to vehicle treated EAE (*p<0.05, ANOVAs; Bonferroni's multiple comparison post-test; n=8-10 mice in each treatment group).

Increased Myelin Thickness and Decreased G Ratio of Callosal Axons in ERβ Ligand treated EAE

Increased number of myelinating cells could lead to improved myelination. Therefore, the degree of myelination was first determined by analyzing myelin by immunohistochemistry. Myelin basic protein (MBP) fluorescence intensity measurements indicated significant callosal demyelination of vehicle treated EAE mice compared to normal (FIG. 4A, B and Supplementary FIG. 1). In contrast, ERβ ligand treated EAE mice had significantly improved myelination that was similar to normal mice (FIG. 4A, B). To assess the integrity of myelination ultrastructure, analysis with electron microscopy by calculating the axon diameter, myelin thickness and mean g ratio of myelinated and unmyelinated axons was performed (FIG. 5). Vehicle treated EAE mice at day 36 of EAE had increased numbers of unmyelinated and thinly myelinated callosal fibers compared to normal mice. Activated microglia and astrocytes present in the CC were accompanied by vacuoles and enlarged mitochondria in axons (FIG. 5A). The CC of ERβ ligand treated EAE mice appeared to have increased numbers of myelinated fibers as compared to vehicle treated EAE mice, with the continued presence of activated microglia and some axons with vacuoles and enlarged mitochondria (FIG. 5A). The most dramatic effect of ERβ ligand treatment was on the myelin sheath thickness. The callosal axons of ERβ ligand treated EAE mice had significantly thicker myelin than vehicle treated mice and occasionally thicker myelin than normal mice (FIG. 5A). Even though there were similar demyelinated regions in the perivascular regions due to continued infiltration, nearby axons in ERβ ligand treated mice had thicker myelin as compared to axons of vehicle-treated mice (FIG. 5B). Quantitative measurement of myelin sheath thickness of all axons within a given field showed nearly 2 fold increase in ERβ ligand treated EAE mice (0.065±0.002 μm) than vehicle treated animals EAE mice (0.027±0.001 μm), and essentially the same thickness as normal mice (0.060±0.002 μm) (FIG. 5Ci). Thus, the g ratio was significantly lower in the ERβ ligand treated EAE CC (0.85±0.012), relative to vehicle-treated EAE CC (0.94±0.026) (p<0.05). The g ratio of ERβ ligand treated EAE mice was similar to that of the normal control group (0.87±0.004-FIG. 5Cii). Scatter plots of the g ratio versus axon diameter highlight the fact that the g ratios were higher in the vehicle treated EAE CC than in the ERβ ligand treated EAE CC (FIG. 5Ciii). Comparing scatter plots of axon diameter versus g ratio or versus axon diameter versus myelin thickness allowed us to identify the cause of g ratio decrease due to increased myelin thickness in the ERβ ligand treated EAE group. Callosal axons of small to medium size showed a more robust increase in myelination with ERβ treatment compared to vehicle-treated EAE or normal controls (FIG. 5Ciii-iv).

As shown in FIG. 4, treatment with an ERβ ligand preserved myelin basic protein immunoreactivity in the CC of mice with EAE.

(A) Brain sections at day 36 after disease induction were post-fixed, immunostained with anti-MBP (red) and imaged at 10× magnification. Vehicle treated mice had reduced MBP immunoreactivity as compared to normal controls, while ERβ ligand treated EAE mice showed relatively preserved MBP staining.

(B) Upon quantification, MBP immunoreactivity in CC was significantly lower in vehicle treated EAE mice as compared to normal mice, while ERβ ligand treated EAE mice demonstrated no significant decreases. Myelin intensity is presented as percent of normal (*p<0.05; **p<0.001, ANOVAs; Bonferroni's multiple comparison post-test; n=8-10 mice in each treatment group).

As shown in FIG. 5, ERβ ligand treated EAE callosal axons have thicker myelin.

(A) Representative electron micrographs of the CC from normal control, vehicle treated EAE and ERβ ligand treated EAE show differential levels of axon myelination (i-iii). Compared to normal controls, the CC of vehicle treated EAE show increased numbers of unmyelinated axons with enlarged mitochondria. ERβ ligand treatment during EAE resulted in a dramatic increase in myelination of mostly smaller axons as compared to vehicle treated EAE and normal control. Pictures are at 4,800× (i) 19,000× (ii), and 48,000× (iii) magnification. Scale bar is 1 μm. (de/un-myelinated axons-↑; thicker myelin sheath ̂; enlarged mitochondria *, vacuoles #).

(B) Additional examples of vehicle treated EAE and ERβ ligand treated EAE callosal axons near a lesion with infiltrating cells. Notice that there are areas in the ERβ ligand treated CC that contain many demyelinating damaged axons similar to those seen extensively in vehicle-treated EAE mice (i). The remaining axons in ERβ ligand treated EAE mice (ii) have thicker myelin sheath compared to vehicle treated EAE mice (iii).

(C) Measurement of myelin thickness showed significant decrease in vehicle treated EAE mice as compared to normal and ERβ ligand treated EAE mice (i). Axon diameter and fiber diameter were measured to further quantify the degree of myelination. Axon diameter/fiber diameter (g ratio) showed a significant increase in vehicle-treated callosal axons and a dramatic decrease in g ratio was observed in ERβ ligand treated EAE callosal axons (ii). Scatter plots of axon diameter versus g ratio (iii) and axon diameter versus myelin thickness (iv) indicated demyelination-induced decreases in myelin thickness in vehicle-treated EAE callosal axons, whereas ERβ ligand-treated EAE mice show increased myelination of small to medium sized callosal axons. The increase in callosal axon g ratio of vehicle treated CC was due to demyelination of axons, whereas the decrease in g ratio in ERβ ligand treated callosal axons was due to an increase in myelination of axons. **p<0.001, *p<0.05, ANOVAs; Bonferroni's multiple comparison post-test. At least 4 mice (36 days post EAE induction) from each group were analyzed and a minimum of 500 fibers were measured from each mouse.

ERβ Ligand Treatment Reduces EAE-Induced Axon Damage and Limits EAE Induced Disorganization of Nodal Proteins in Callosal Axons

Chronic EAE induced demyelination is accompanied by significant axon damage which could theoretically be reversed by the increased axon myelination observed in ERβ ligand treated EAE mice. Decreased axon damage during EAE was confirmed by performing immunohistochemistry with neurofilament (NF200), a common axon marker, and beta amyloid precursor protein (β-APP) a marker of axon damage. In normal control, NF200 was visible in small areas (likely nodes of Ranvier) of myelinated axons that were co-stained with MBP (FIG. 6Ai). Further, there was no significant β-APP immunoreactivity thereby indicating intact, healthy axons (FIG. 6Bi, C). In contrast, vehicle treated EAE axons had large areas of NF200 positivity and minimal MBP staining denoting demyelination (FIG. 6Aii). In addition, these demyelinated axons showed β-APP immunoreactive axonal swelling, axon bulbs and transected axons in the CC white matter (FIG. 6Bii, C). Callosal axons of ERβ ligand treated EAE mice show less demyelination and reduced amount of β-APP immunoreactivity than vehicle treated EAE mice (FIG. 6A-Biii, C).

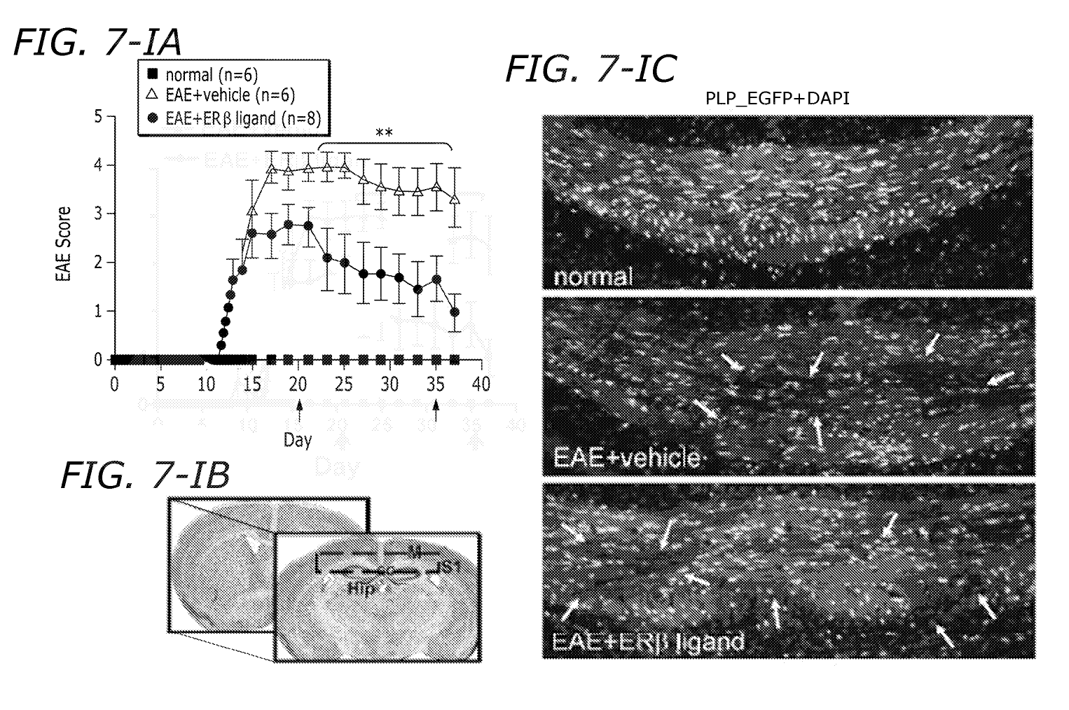

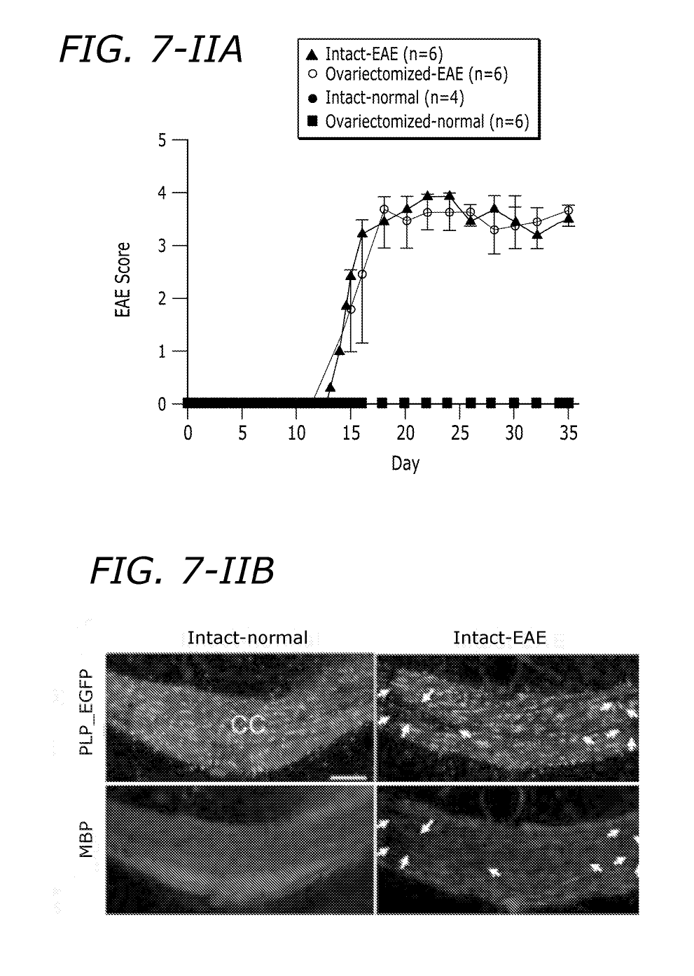

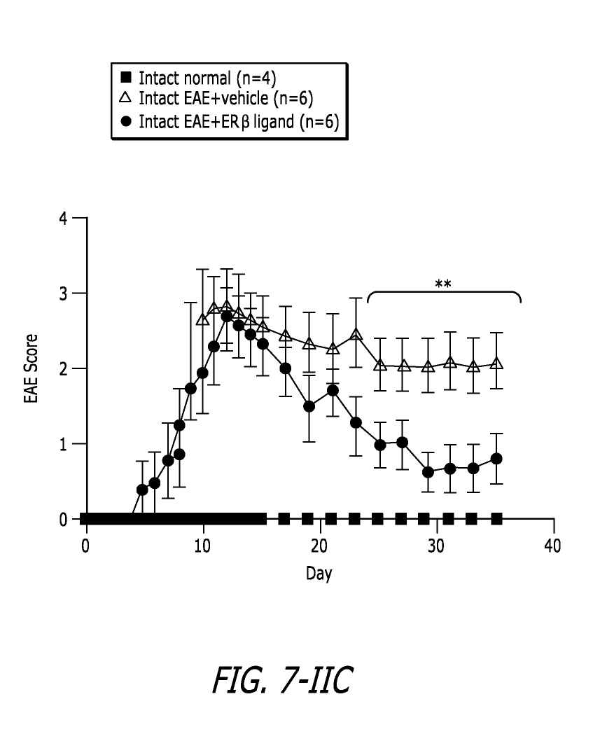

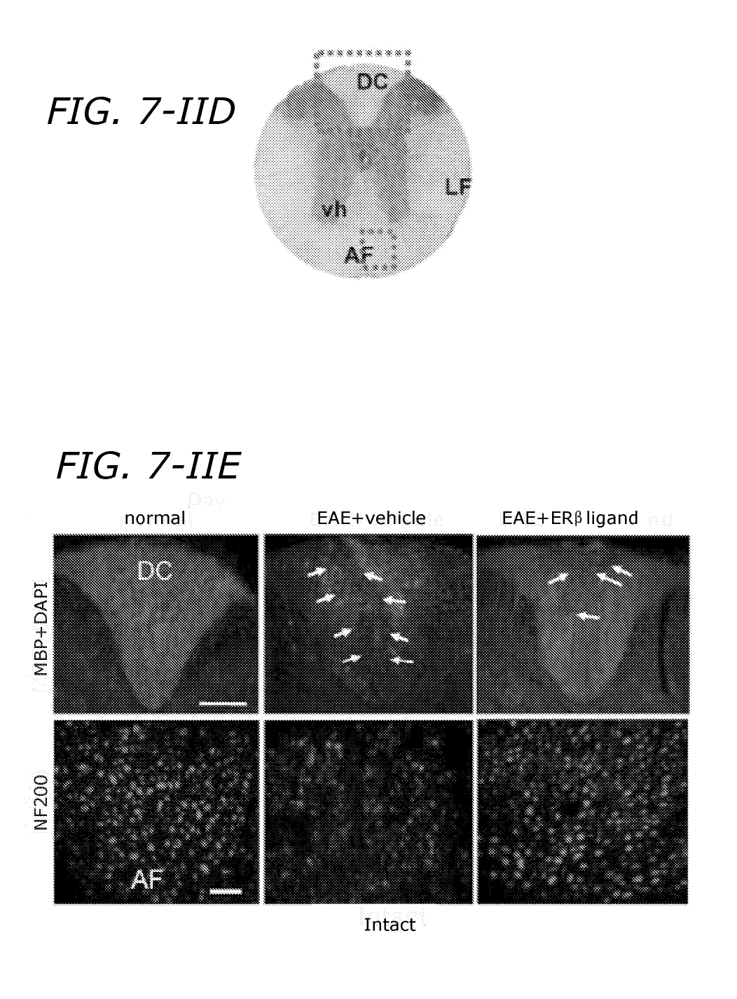

Saltatory conduction of myelinated axons depends on the presence of nodes of Ranvier on healthy axons. Demyelination leading to nodal disorganization and axon damage is prominent in MS lesions and is likely a major cause of conduction failure. Similar nodal disorganization and conduction failure has been observed in EAE spinal cord. Therefore, the effect of EAE-induced demyelination and ERβ ligand treatment-induced hypermyelination on nodal proteins was analyzed in the CC. Nodal regions were identified and delineated with antibodies against Caspr, a component of axo-glial junctions that appears paranodally. In the CC of normal mice, Nav1.6⁺ staining was found mostly between Caspr⁺ staining, clearly identifying nodes of Ranvier (FIG. 7A). During chronic EAE, Caspr staining levels were decreased significantly to less than 60% of normal CC (FIG. 7B). Surprisingly, intact Caspr pairs contained Nav1.6 at the nodes, similar to normal CC. The remaining Nav1.6 protein instead of being concentrated between Caspr pairs had become diffuse over the length of the axons (FIG. 7A).

Kv1.2 potassium channel proteins appear as juxtaparanodal pairs in normal myelinated axons (FIG. 7C). Demyelination in vehicle treated EAE was associated with increased expression of Kv1.2 and a lengthening of Kv1.2 immunostaining across the entire axon length. ERβ ligand treated EAE callosal axons had only a few areas of diffuse Kv1.2 staining, but overall showed near normal levels of juxtaparanodal Kv1.2 staining (FIG. 7C).

As shown in FIG. 6, a decrease in demyelination and axon damage in ERβ ligand treated EAE callosal axons.

(A) High magnification confocal images (60×) were taken to identify the presence of demyelination and axon damage. Normal myelinated axons had even MBP immunostaining with small areas that were MBP⁻ and NF200⁺ and are most likely the nodes of Ranvier (↑). Vehicle treated EAE axons expressed large areas that were MBP⁻]and NF200⁺ indicative of demyelination (*). ERβ ligand treatment during EAE had myelinated axons similar to normal.

(B) Axon degeneration was assessed with beta amyloid precursor protein (β-APP) accumulation. Unlike the normal control CC that did not show axonal pathology with β-APP⁻(blue) immunostaining, vehicle treated EAE mice had demyelinated axons that showed swelling, beading (̂) and increased areas of β-APP accumulation. ERβ treatment during EAE significantly reduced the extent of axon pathology.

(C) Quantification of β-APP immunostaining intensity in the CC showed nearly 70% less accumulation in ERβ ligand treated EAE compared to vehicle treated EAE. (*p<0.05; **p<0.001, ANOVAs; Bonferroni's multiple comparison post-test; n=5 mice in each treatment group).

As shown in FIG. 7, ERβ ligand treatment limits EAE induced disorganization of nodal proteins in callosal axons.

(A) CC sections were immunostained with nodal proteins Caspr (red, marked with white arrow heads) and Nav1.6 (green). A significant decrease in Caspr and Nav1.6 staining occurred in the CC of vehicle treated EAE mice. In addition, extensive regions of axons (white stars) were immunostained with Nav1.6 not confined between Caspr pairs. ERβ ligand-treated EAE CC axons contained Caspr pairs with Nav1.6 similar to normal control. Note: PLP_EGFP-green channel was dropped and Nav1.6 immunostaining performed with TRITC conjugated secondary was pseudo-colored to green for clarity.

(B) Quantification of Caspr protein pairs alone and Caspr protein pair encompassing Nav1.6 protein showed a significant decrease in vehicle treated EAE callosal axons compared to those of normal and ERβ ligand treated EAE (*p<0.05, **p<0.001, ANOVAs; Bonferroni's multiple comparison post-test; n=5 mice in each treatment group).

(C) Juxtaparanodal Kv1.2 protein (red, arrowheads) immunostaining increased in the CC of vehicle treated EAE mice. Specifically, Kv1.2 immunostaining was obvious throughout the length of some axons (white stars). No significant difference was observed in ERβ ligand treated EAE axons compared to normal.

ERβ Ligand Treatment During EAE Restores Callosal Conduction, Axon Velocity, and Axon Refractoriness of Callosal Axons

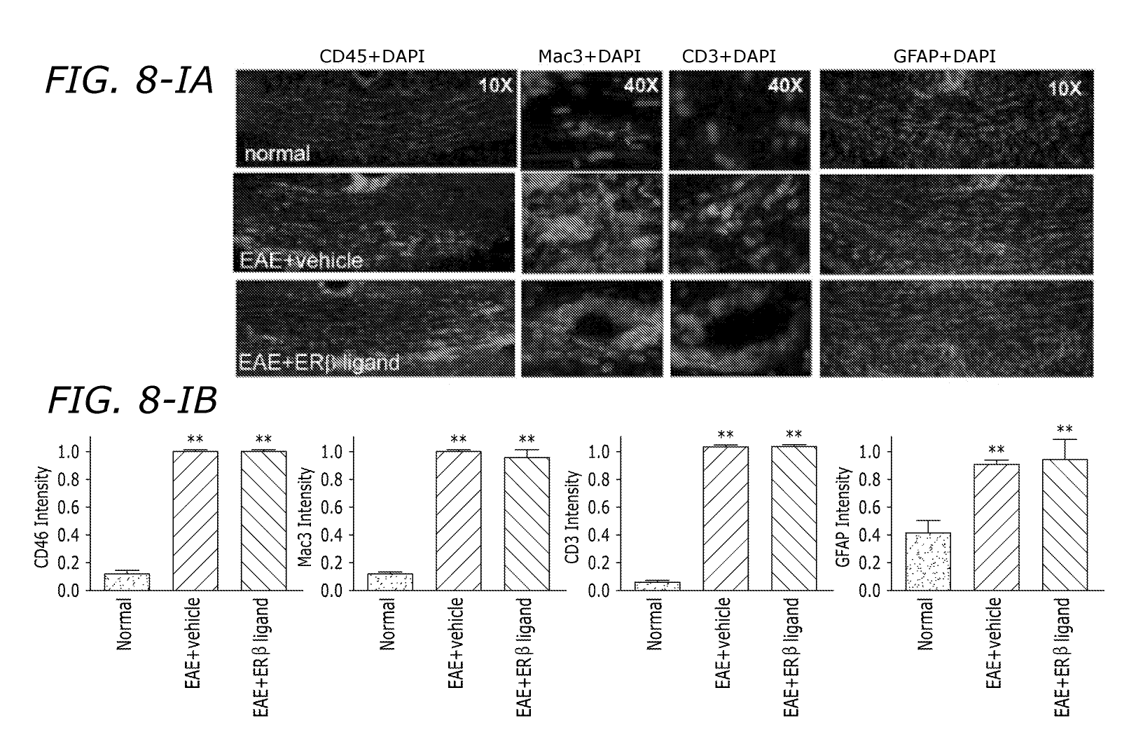

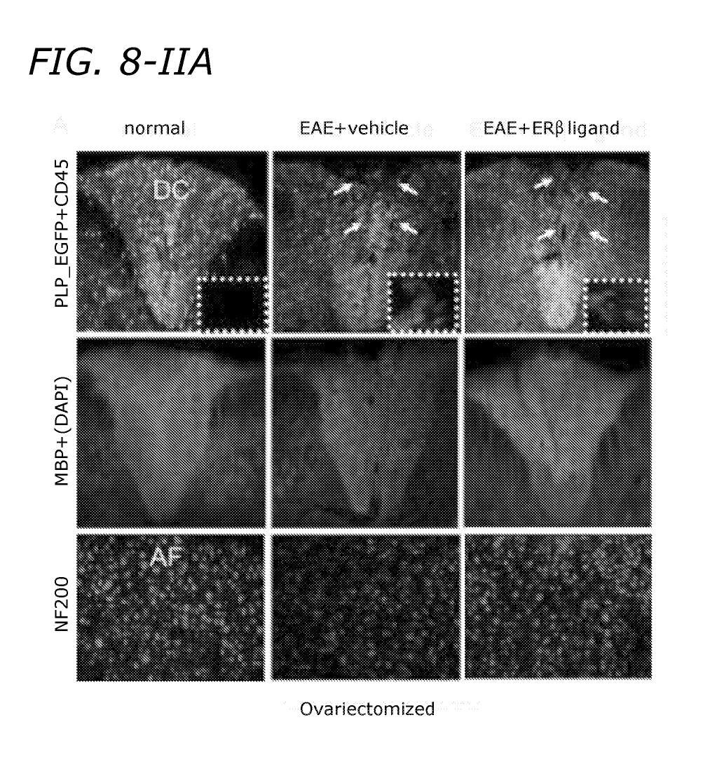

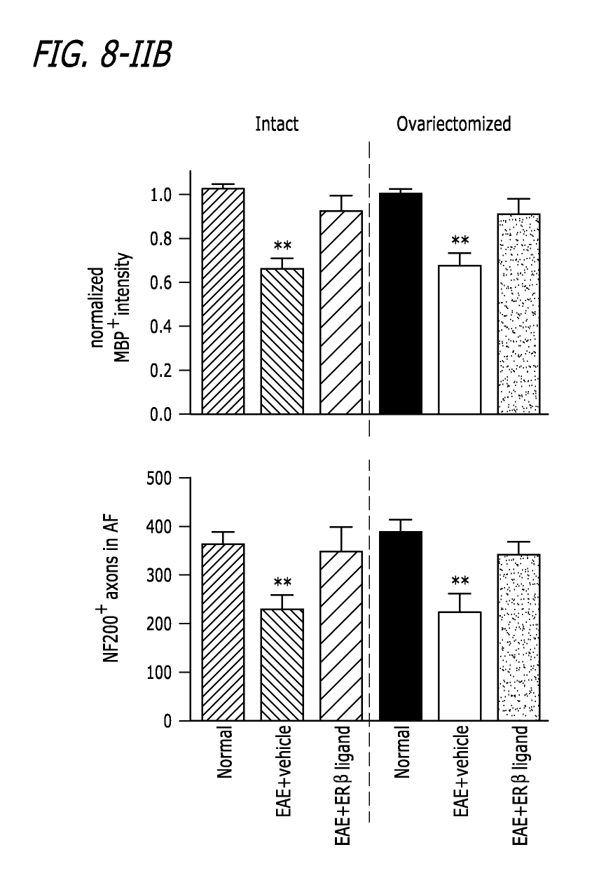

Callosal axons play a major role in interhemispheric transfer and integration of sensorimotor and cognitive information. To characterize the functional consequences of the neuropathology in the CC during EAE, compound action potentials (CAPs) were recorded in callosal axons (FIG. 8). Coronal brain slices with midline-crossing segments of the CC, corresponding approximately to plates 29-48 in the atlas of Paxinos and Franklin, were used for recording. Two downward phases of the CAPs ‘N1’ and ‘N2’ were observed, likely representing fast depolarization from large, myelinated axons and slower depolarization from non-myelinated axons, respectively. Typical voltage traces are shown in FIG. 8B. During early EAE (day 20), both N1 and N2 CAP amplitudes were decreased to nearly 50% of normal (p<0.001, FIG. 8C-D). This decrease persisted later into EAE (day 36). Treatment with ERβ ligand during EAE induced an increase in N1 and N2 compared to vehicle-treated mice, which was a trend when examined early, but became significant when examined late (p<0.05, FIG. 8D).

The myelinated CAP component, N1 of ERβ ligand treated EAE callosal axons showed a small but significant shift to the left of vehicle treated EAE callosal axons (FIG. 8B). A shift to the left could theoretically be due to an increase in axon conduction velocity as a consequence of improved myelination. To confirm this we first measured conduction velocity of EAE callosal axons in the absence and presence of ERβ ligand treatment as previously described. (Crawford , et al., “Assaying the functional effects of demyelination and remyelination: revisiting field potential recordings.” J Neurosci Methods 2009a; 182: 25-33). The peak latency of the N1 and N2 components were measured and graphed versus distance. Linear regression analysis was performed for each CAP component to yield a slope that is the inverse of the velocity, followed by statistical comparison of the velocities. The conduction velocity of the N1 component for normal callosal axons was 1.82±0.15 m s⁻¹. Whereas, the N1 conduction velocity of vehicle treated EAE was decreased to 1.69±0.10 m s⁻¹. ERβ ligand treatment during EAE induced an increase in conduction velocity to 1.92±0.11 m s⁻¹, a significant increase compared to both vehicle-treated EAE and normal group. The conduction velocity of N2 component was not different between normal and treatment groups and was 0.57±0.012 (normal), 0.55±0.20 (vehicle-treated EAE), and 0.56±0.10 (ERβ ligand treated EAE) m s⁻¹ respectively. In conclusion ERβ ligand treated EAE callosal axons showed a slight but significant improvement in conduction velocity.

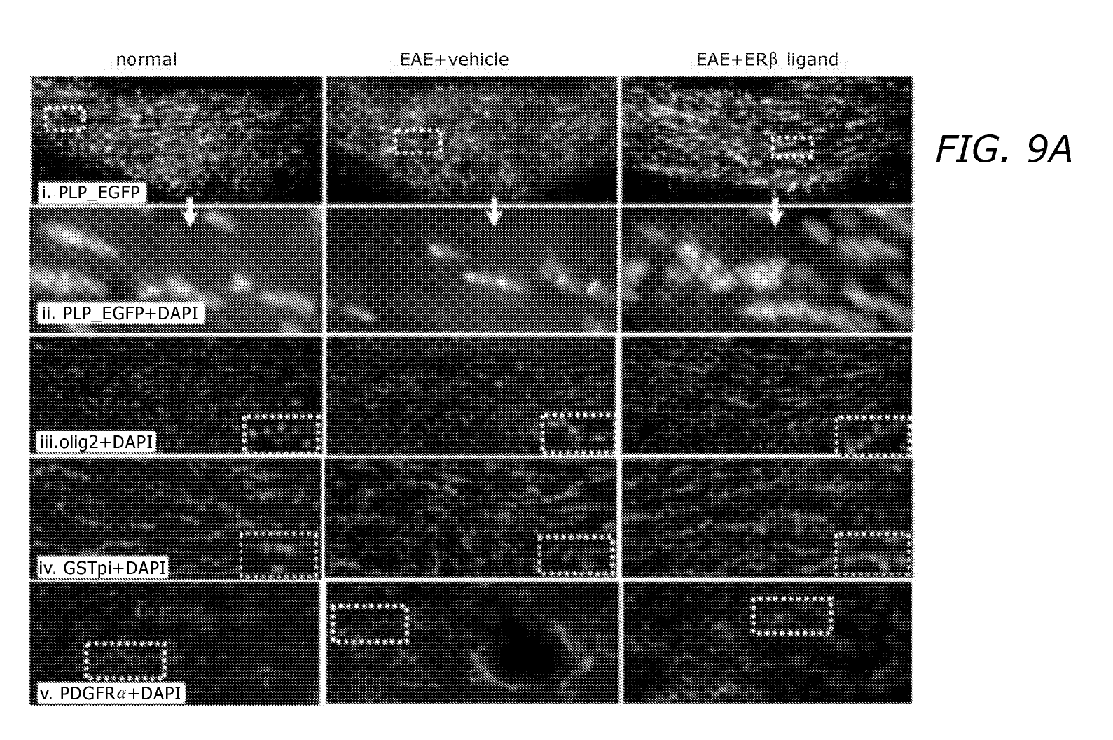

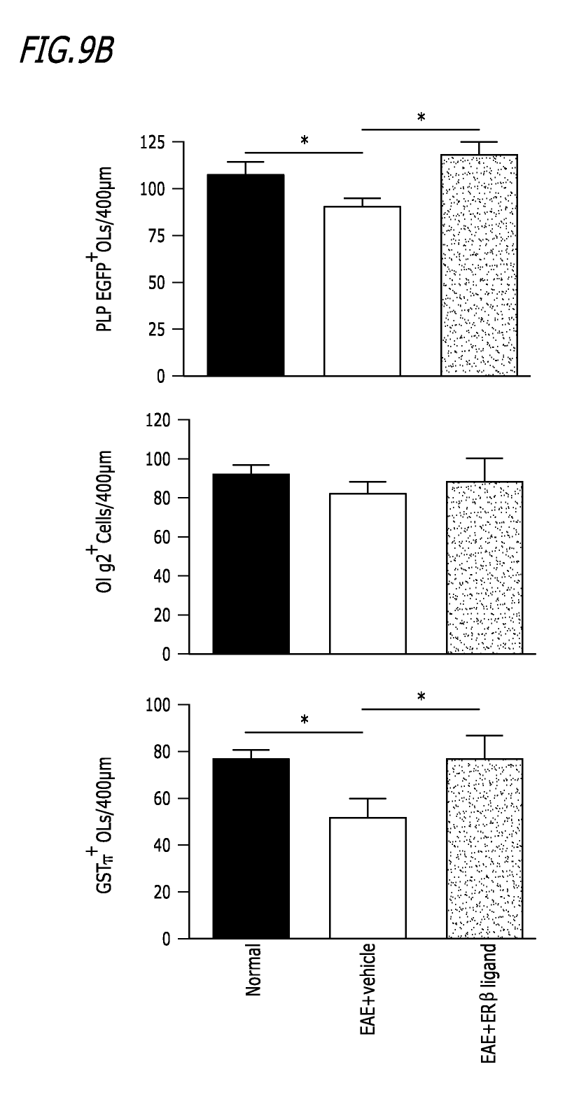

Chronic EAE-induced demyelination and conduction deficit is also accompanied by functional axon deficit. Axonal deficits were estimated by assaying changes in axon refractoriness. FIG. 9A shows an example series of the second response evoked in paired stimulus presentations, after subtracting out the response to a conditioning pulse. Traces shown are for normal, vehicle treated EAE and ERβ ligand treated EAE mice at interpulse intervals from 2 to 8 ms. The CAP component-amplitude elicited by the second pulse in each paired stimulation (C₂) divided by the CAP component-amplitude to single pulse stimulation (C₁) was plotted. These C₂/C₁ ratios were averaged for each analytic group and mean values fitted to Boltzmann sigmoid curves. Rightward shifts in these curves correspond to increases in the refractory recovery cycle in the callosal axons and are indicative of functional axonal deficit

In the normal group, the N1 component evoked by the second of a pair of pulses was 50% of the amplitude of a single pulse presentation when the interpulse interval was 2.2±0.21 ms. The interpulse interval for vehicle treated EAE had slower responses of 3.9±0.15 ms. ERβ ligand treated callosal EAE axons had an interpulse interval of 3.0±0.11 ms (FIG. 9B), significantly better than the interpulse interval of vehicle-treated EAE callosal axons. The interpulse intervals for the N2 component of all three groups were not significantly different at 3.1±0.10 ms (normal), 3.5±0.05 ms (vehicle treated EAE), and 3.1±0.16 ms (ERβ ligand treated EAE).

As shown in FIG. 8, treatment with ERβ ligand restores callosal conduction of both myelinated and non-myelinated axons of mice with EAE.

(A) Compound action potential (CAP) responses were recorded from slices with midline-crossing segments of the CC overlying the mid-dorsal hippocampus. Stimulating (Sti) and recording (Rec) electrodes were each placed −1 mm away from midline. (CC: corpus callosum; Hip: hippocampus; S1: somatosensory cortex; M: motor cortex).

(B) Typical CC CAPs from normal-black, vehicle treated EAE-red, and ERβ ligand treated EAE-blue brain slices evoked (at a stimulus of 4 mA) at day 36 after disease induction. There is a decrease in N1 and N2 amplitude in the vehicle treated EAE group. Treatment with ERβ ligand during EAE induced a latency shift in N1 peak, as well as a muted decrease in N1 and N2 CAP amplitude compared to vehicle alone. (Dashed vertical line represents CAPs beyond the stimulus artifact.)

(C-D) Quantification of N1 and N2 CAP amplitudes in the CC of vehicle treated EAE mice showed a significant decrease early, at day 20, and late, at day 36 after disease induction in disease. ERβ ligand treatment showed a significant improvement in CAP response late in disease. Number of mice=4 per treatment group, number of CC sections per mouse=3, total number of sections per treatment group=12. Statistically significant compared with normal at 2-4 mA stimulus strength (*p<0.05; **p<0.001; ANOVAs; Bonferroni's multiple comparison post-test).

As shown in FIG. 9, treatment with ERβ ligand restores refractoriness of callosal axons.

(A) Example waveforms shows the second response in paired stimuli after subtraction of the response to the conditioning pulse (interpulse intervals=2-8 ms) for normal, vehicle treated EAE and ERβ ligand-treated EAE callosal axons at later time point. (Dashed vertical line represents CAPs beyond the stimulus artifact.)

(B) Average C₂/C₁ ratios [obtained from plots of mean CAP amplitude elicited by the second pulse in each paired stimulation (C₂) divided by the CAP amplitude to single pulse stimulation (C₁)] were fitted to Boltzmann sigmoid curves. A rightward shift in curves for N1 shows decreased refractoriness in vehicle-treated and ERβ ligand-treated EAE groups (n=4). ERβ ligand-treated EAE callosal axons show a significant increase (a leftward shift in the curve compared to vehicle treatment alone) in refractoriness of N1 compared to those with vehicle treatment alone. The interpulse interval values (mean±SD) of N1 and N2 component for normal, vehicle treated EAE and ERβ ligand treated EAE callosal axons are presented in the table.

Callosal and Corticospinal Tracts are Preserved During ERA Ligand Treatment

Finally, to assess the extent of EAE-induced axon degeneration and the effects of ERβ ligand treatment during EAE; the callosal tracts were evaluated by neuronal tract tracing studies. Using a precise micro injector, each group of mice were injected with the tract dye, dextran red (10,000 MW) in the right hemisphere. The injection site was the primary motor and sensorimotor cortex near layer II-V to label the pyramidal neurons, thereby establishing a direct labeling method to evaluate these axon tracts.

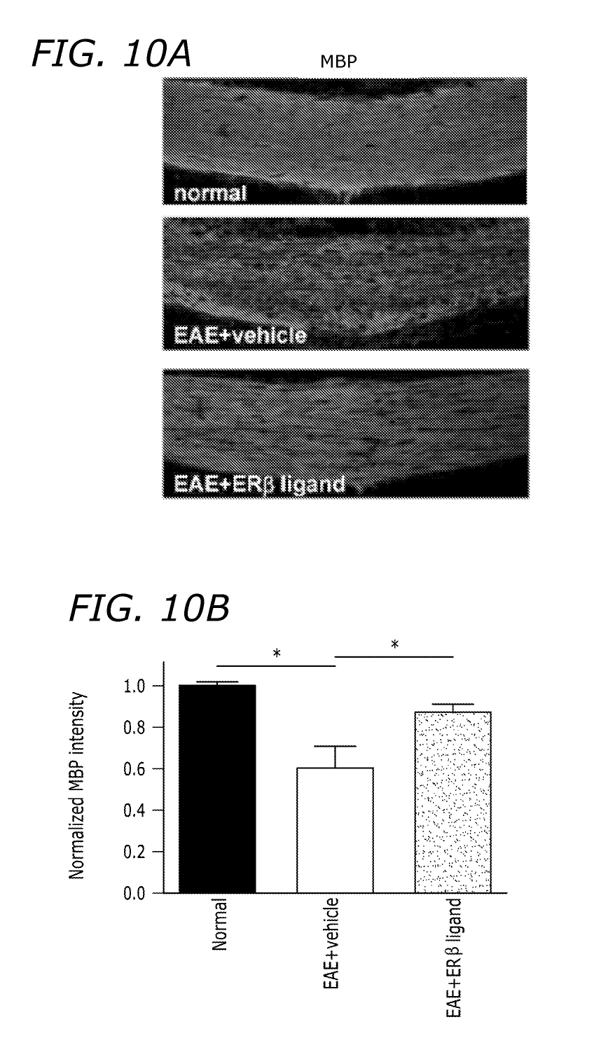

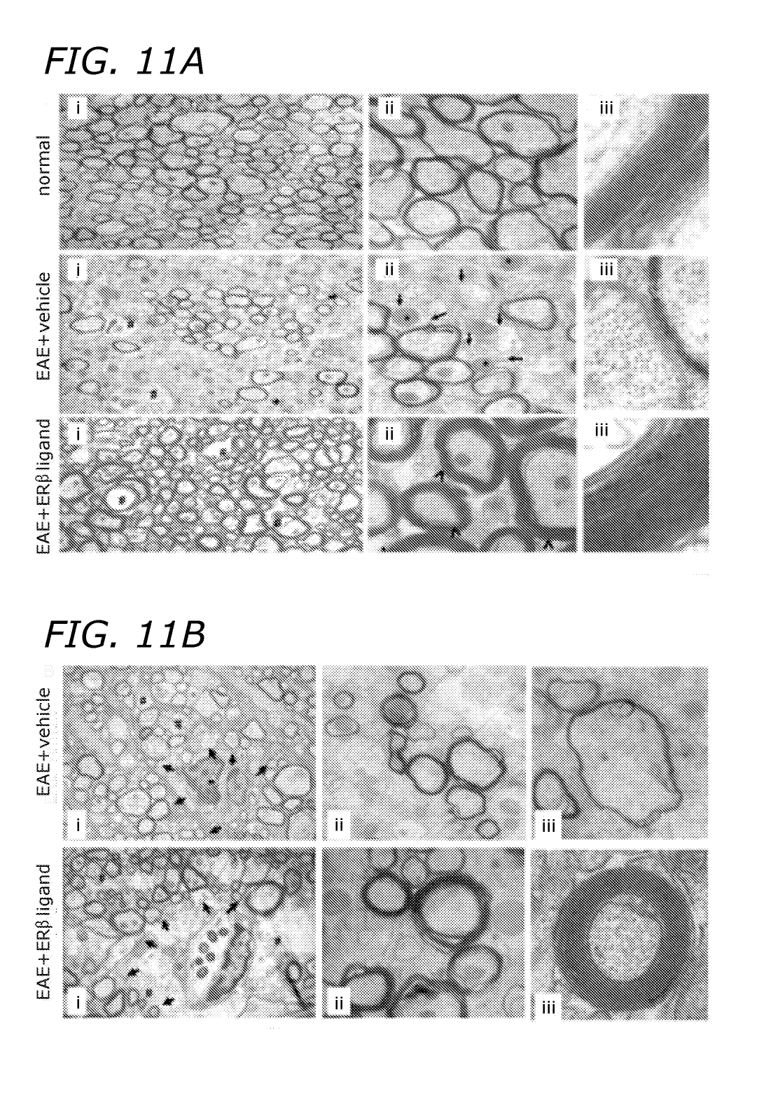

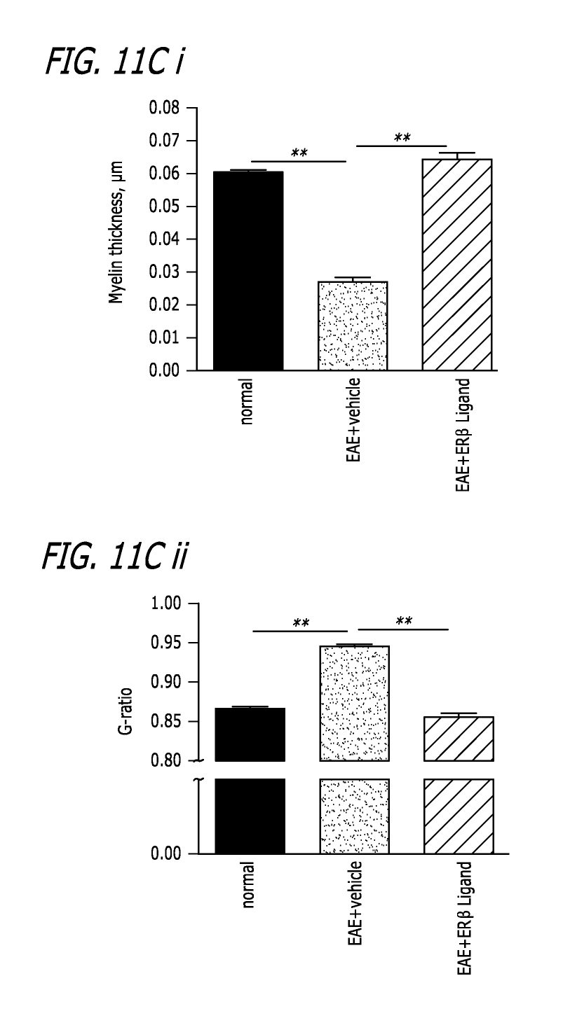

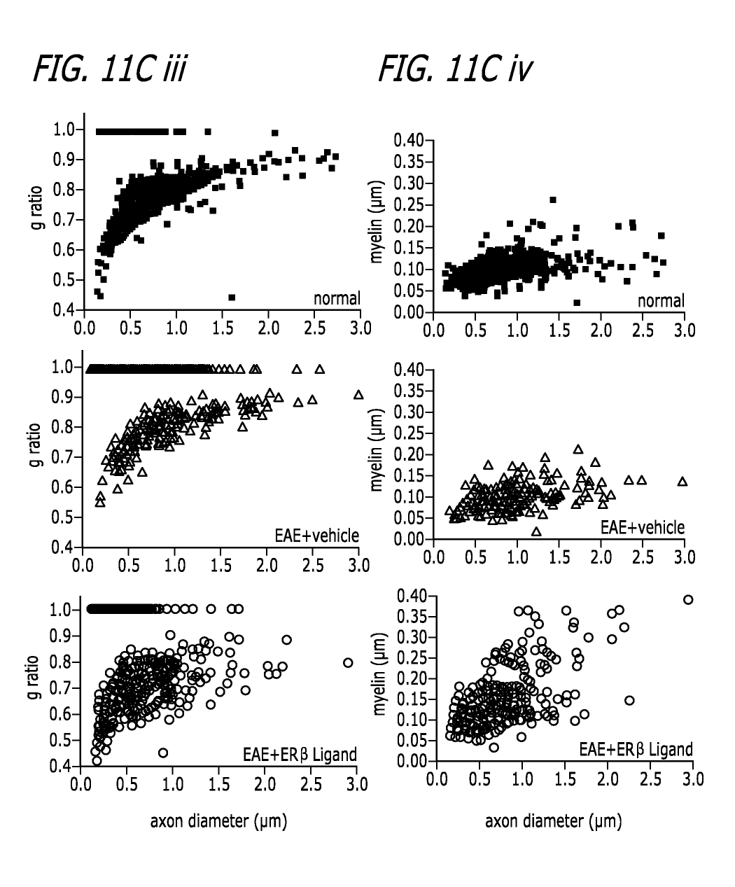

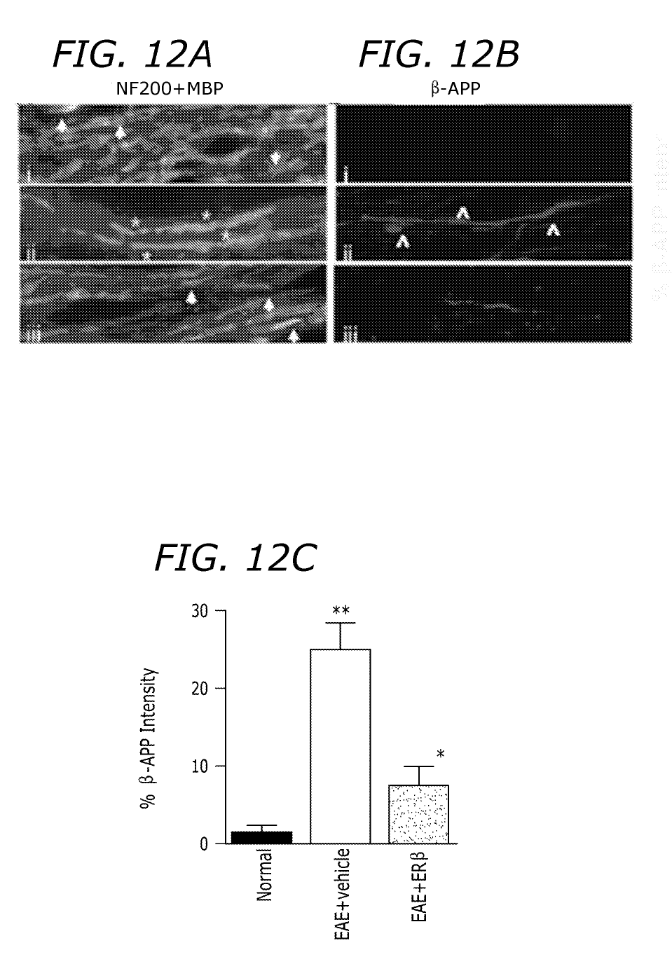

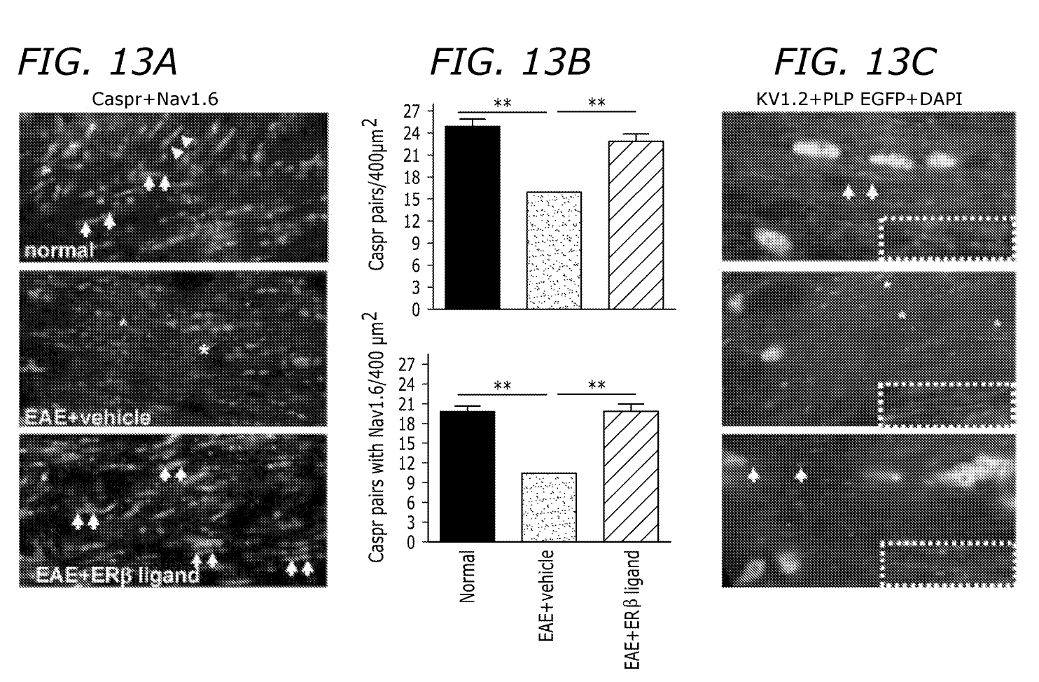

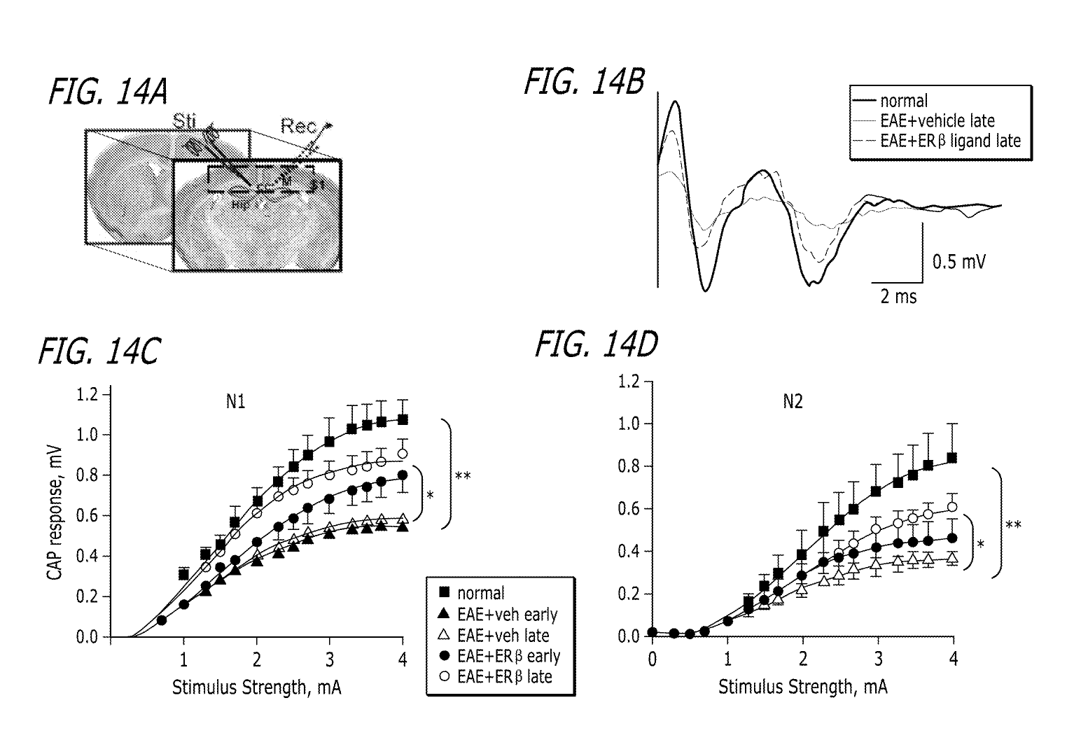

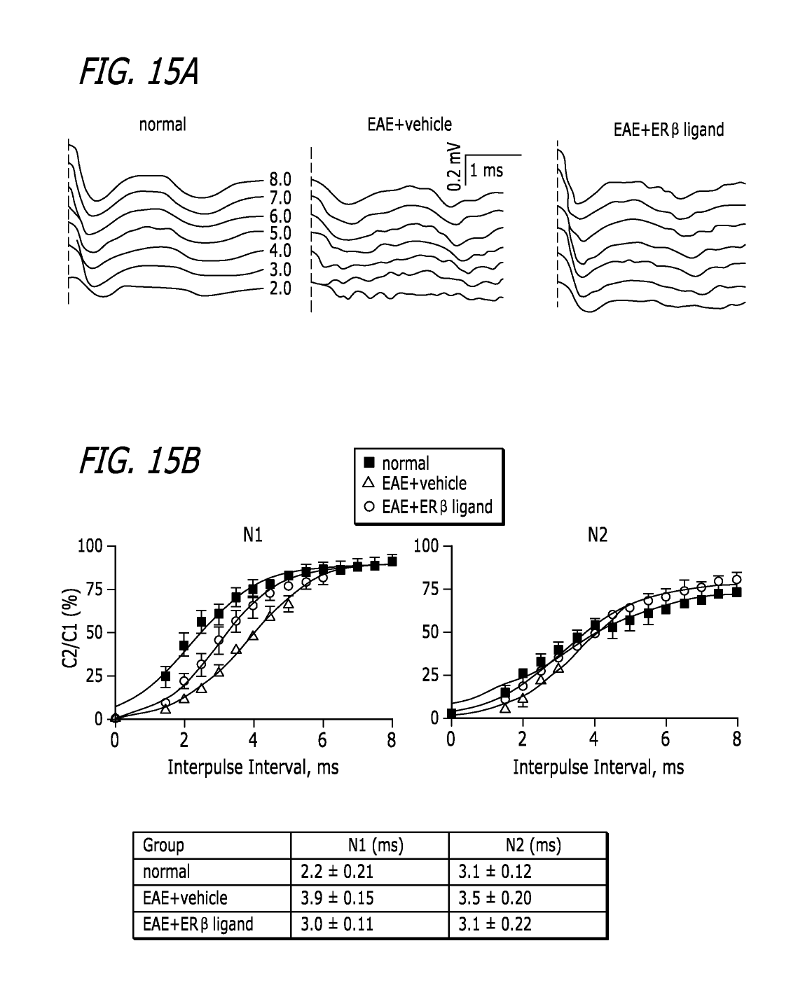

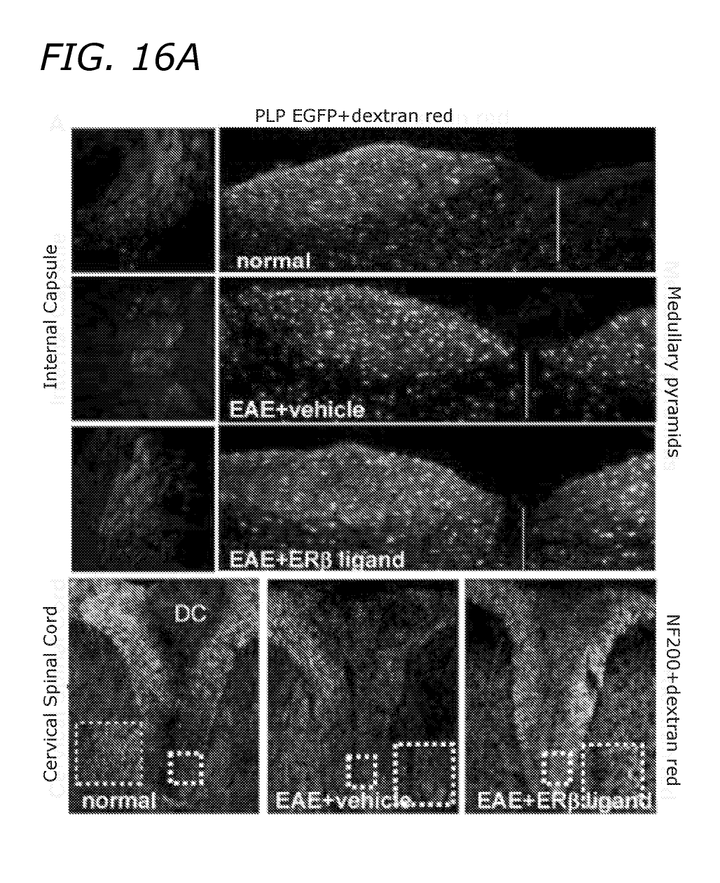

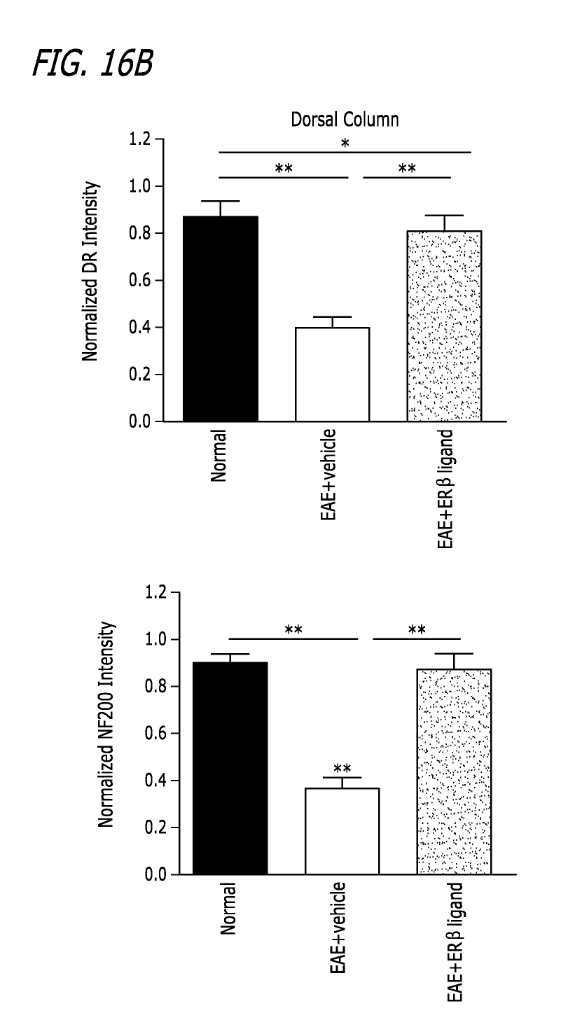

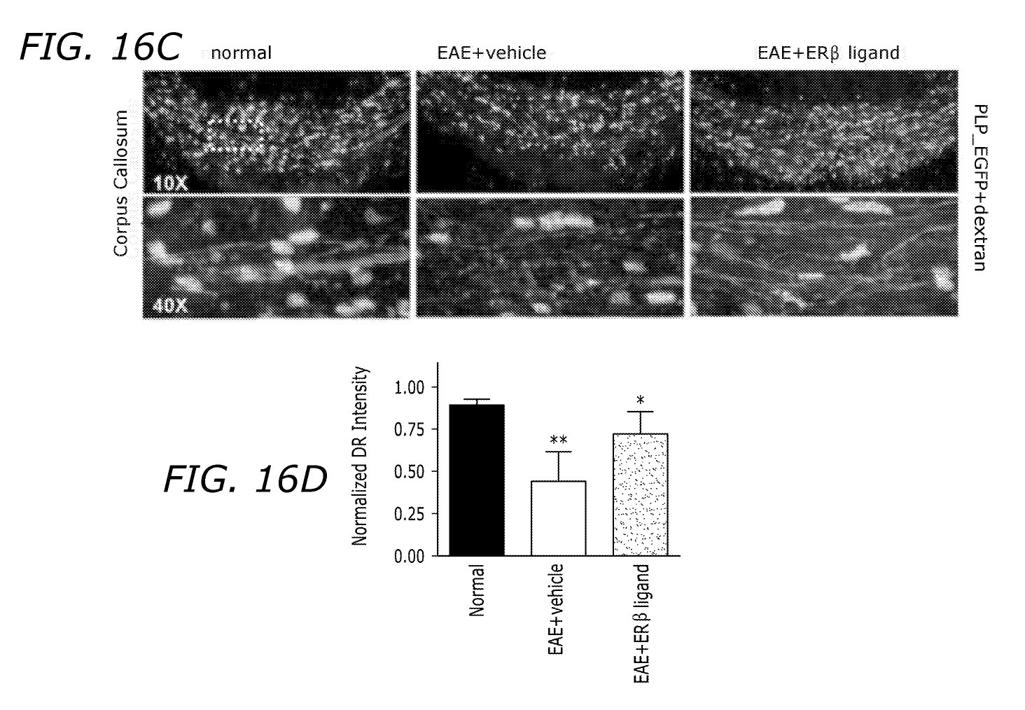

Previous studies have shown a disruption of Dil-dye labeled corticospinal (CST) axonal damage in spinal cord of EAE mice. We confirmed our method of labeling by first analyzing the EAE-CST tract. In the rodent, the only neurons in the forebrain that send axons to the spinal cord are those of the CST through the internal capsule and medullary pyramid. Most of the CST decussates to the opposite side in the medulla oblongata and descends in the most-ventral part of spinal dorsal funiculus. Unilateral labeling of the CST located in the internal capsule, medullary pyramids and at the ventral aspect of the cervical dorsal columns in the cord was clearly visible from normal mice. These regions were labeled discretely by dextran red fluorescence and their individual axons were identifiable (FIG. 10A). However, compared to normal controls, vehicle treated EAE mice had reduced and discontinuous tract dye staining, indicating dysfunction in the CST tract. The ERβ ligand treated EAE group had significantly improved dye staining as compared to vehicle treated EAE mice (FIG. 10A). Very few dye-filled discontinuous and swollen axon varicosities were present in the ERβ ligand treated animals. Quantification of dextran red dye or NF200⁺ axon intensity showed a significant decrease in the dorsal column during vehicle treatment, whereas ERβ ligand treatment showed similar staining as normal (FIG. 10B).

Dextran red labeled axons from layer II/III and layer V descend and cross in the CC (FIG. 10C). In normal controls, bundles of axons that started from the right side of CC were labeled with dextran red and crossed over to the left hemisphere. Comparatively, fewer labeled axons crossed over to the left hemisphere in the vehicle treated EAE mice. Here, the dye fluorescence was punctate and discontinuous, indicative of axon transport deficits. In contrast, ERβ ligand treated EAE mice showed much better labeling as compared to vehicle treated EAE. Nearly 80% of callosal axons in ERβ ligand treated EAE animals were labeled and very few axons showed punctate dye accumulation (FIG. 10C-D).

As shown in FIG. 10, ERβ ligand treatment prevented corticospinal tract (CST) and callosal pathology induced by EAE.

(A) The CST from layer II/III and layer V neurons were followed through the internal capsule (dextran red only), medullary pyramids (dextran red and PLP_EGFP) and the spinal cord (dextran red and NF200) in the ventral-most part of the dorsal column (DC). Dextran red labeling was decreased in these areas in the vehicle treated EAE compared to those of normal. ERβ ligand-treated EAE showed improvement, especially in the high cervical spinal cord. Fluorescent red axons were seen only in one side and the axon intensity was measured from single confocal images of high cervical spinal cord. At the cervical level, the dextran labeled axon number of vehicle treated EAE mice was significantly decreased compared with normal mice, while the ERβ ligand-treated EAE axons showed increased numbers similar to normal controls.

(B) Cervical spinal cord sections from normal, vehicle treated and ERβ ligand treated EAE animals that were injected with dextran red were co-immunostained with neurofilament marker NF200 (green). Dorsal column was delineated and dextran red and NF200 fluorescence intensity were calculated and normalized to normal. Vehicle treated EAE dorsal column showed a significant decrease in dextran red and NF200 fluorescence, whereas ERβ ligand treated EAE dorsal column had similar levels as normal (*p<0.05; **p<0.001, ANOVAs; Bonferroni's multiple comparison post-test; n=5).

(C) Representative fluorescent images show callosal tracts of normal, vehicle treated EAE and ERβ ligand-treated EAE animals 7 days post-dextran red injection. Normal CC shows green PLP_EGFP⁺ cells and intense, coherent dextran red labeling of callosal axons. The CC of vehicle treated EAE mice had decreased PLP_EGFP⁺ cells, as well as decreased, punctate and discontinuous dextran red labeling. ERβ ligand-treated EAE had many more PLP_EGFP⁺ cells and increased number of axons that were dextran red labeled compared to vehicle treated EAE animals. Scale bar is 100 μm.

(D) Quantification of dextran red intensity in known CC regions indicated a significant decrease during vehicle treated EAE compared to normal. ERβ ligand treated EAE mice were not significantly different than normal control. (*p<0.05; **p<0.001, ANOVAs; Bonferroni's multiple comparison post-test; n=4).

What is claimed is:

1. A method for reducing the clinical symptoms of a neurodegenerative disease in a mammal, comprising administering to the mammal a therapeutically effective dose of at least one of an estrogen receptor beta ligand or an interferon beta.

2. The method of claim 1, wherein the beta-interferon is interferon-β 1a or interferon-β 1b.

3. The method of claim 1, wherein the neurodegenerative disease is multiple sclerosis.

4. The method of claim 1, wherein the beta-interferon is selected from the following or the active ingredient therein: Avonex at a dosage of about 30 mcg once a week, Rebif at a dosage of about 22-44 mcg three times a week, or Betaseron at a doasage of about 0.25 mg every other day.

5. The method of claim 1, wherein the beta-interferon is selected from the following or the active ingredient therein: Avonex at about 15-29 mcg, Rebif at about 11-21 mcg, or Betaseron at about 0.125-0.24 mg.

6. The method of claim 1, wherein the estrogen receptor beta ligand is diarylpropionitrile or estriol selected at a dose of: about 2-16 mg/kg/day, about 4-12 mg/kg/day, or about 8 mg/kg/day.

7. A method for providing neuronal protection in a mammal afflicted with a neurodegenerative disease, comprising administering to the mammal a therapeutically effective dose of at least one of an estrogen receptor beta ligand or an interferon beta.

8. The method of claim 7 wherein the neuronal protection comprises the preservation of spinal cord axons.

9. The method of claim 7, wherein the beta-interferon is interferon-β 1a or interferon-β 1b.

10. The method of claim 7, wherein the neurodegenerative disease is multiple sclerosis.

11. The method of claim 7, wherein the beta-interferon is selected from the following or the active ingredient therein: Avonex at a dosage of about 30 mcg once a week, Rebif at a dosage of about 22-44 mcg three times a week, or Betaseron at a doasage of about 0.25 mg every other day.

12. The method of claim 7, wherein the beta-interferon is selected from the following or the active ingredient therein: Avonex at about 15-29 mcg, Rebif at about 11-21 mcg, or Betaseron at about 0.125-0.24 mg.

13. The method of claim 7, wherein the estrogen receptor beta ligand is diarylpropionitrile or estriol selected at a dose of: about 2-16 mg/kg/day, about 4-12 mg/kg/day, or about 8 mg/kg/day.

14. A method for preserving myelinating oligodendrocytes in a mammal afflicted with a neurodegenerative disease, comprising administering to the mammal a therapeutically effective dose of at least one of an estrogen receptor beta ligand.

15. The method of claim 14, wherein the neurodegenerative disease is multiple sclerosis.

16. The method of claim 14, wherein the estrogen receptor beta ligand is diarylpropionitrile or estriol selected at a dose of: about 2-16 mg/kg/day, about 4-12 mg/kg/day, or about 8 mg/kg/day.

17. A method for preserving axon myelination in a mammal afflicted with a neurodegenerative disease, comprising administering to the mammal a therapeutically effective dose of at least one of an estrogen receptor beta ligand.

18. The method of claim 17, wherein the neurodegenerative disease is multiple sclerosis.

19. The method of claim 17, wherein the estrogen receptor beta ligand is diarylpropionitrile or estriol selected at a dose of: about 2-16 mg/kg/day, about 4-12 mg/kg/day, or about 8 mg/kg/day.

20. A method for stimulating axon remyelination in a mammal afflicted with a neurodegenerative disease, comprising administering to the mammal a therapeutically effective dose of at least one of an estrogen receptor beta ligand.

21. The method of claim 20, wherein the neurodegenerative disease is multiple sclerosis.

22. The method of claim 20, wherein the estrogen receptor beta ligand is diarylpropionitrile or estriol selected at a dose of: about 2-16 mg/kg/day, about 4-12 mg/kg/day, or about 8 mg/kg/day.

23. A method for reducing nervous system inflammation in a mammal, the method comprising the steps of administering to the mammal a therapeutically effective dose of at least one of an estrogen receptor beta ligand or an interferon beta.

24. The method of claim 23, wherein the beta-interferon is interferon-β 1a or interferon-β 1b.

25. The method of claim 23, wherein the nervous system inflammation results from multiple sclerosis.

25. The method of claim 23, wherein the beta-interferon is selected from the following or the active ingredient therein: Avonex at a dosage of about 30 mcg once a week, Rebif at a dosage of about 22-44 mcg three times a week, or Betaseron at a doasage of about 0.25 mg every other day.

26. The method of claim 23, wherein the beta-interferon is selected from the following or the active ingredient therein: Avonex at about 15-29 mcg, Rebif at about 11-21 mcg, or Betaseron at about 0.125-0.24 mg.

27. The method of claim 23, wherein the estrogen receptor beta ligand is diarylpropionitrile or estriol selected at a dose of: about 2-16 mg/kg/day, about 4-12 mg/kg/day, or about 8 mg/kg/day.

28. A method for reducing the expression of VLA-4 on CD4-type T cells in a mammal afflicted with a neurodegenerative disease, comprising the steps of administering to the mammal a therapeutically effective dose of at least one of an estrogen receptor beta ligand or an interferon beta.

29. The method of claim 28, wherein the beta-interferon is interferon-β 1a or interferon-β 1b.

30. The method of claim 28, wherein the nervous system inflammation results from multiple sclerosis.

31. The method of claim 28, wherein the beta-interferon is selected from the following or the active ingredient therein: Avonex at a dosage of about 30 mcg once a week, Rebif at a dosage of about 22-44 mcg three times a week, or Betaseron at a doasage of about 0.25 mg every other day.

32. The method of claim 28, wherein the beta-interferon is selected from the following or the active ingredient therein: Avonex at about 15-29 mcg, Rebif at about 11-21 mcg, or Betaseron at about 0.125-0.24 mg.

33. The method of claim 28, wherein the estrogen receptor beta ligand is diarylpropionitrile or estriol selected at a dose of: about 2-16 mg/kg/day, about 4-12 mg/kg/day, or about 8 mg/kg/day.

34. A method for reducing IL-17 levels in a mammal afflicted with an infiltrating immune system response, the method comprising the steps of administering to the mammal a therapeutic amount of a primary agent being an estrogen receptor beta ligand and a secondary agent being interferon beta.

35. The method of claim 34, wherein the beta-interferon is interferon-β 1a or interferon-β 1b.

36. The method of claim 34, wherein the nervous system inflammation results from multiple sclerosis.

36. The method of claim 34, wherein the beta-interferon is selected from the following or the active ingredient therein: Avonex at a dosage of about 30 mcg once a week, Rebif at a dosage of about 22-44 mcg three times a week, or Betaseron at a doasage of about 0.25 mg every other day.

38. The method of claim 34, wherein the beta-interferon is selected from the following or the active ingredient therein: Avonex at about 15-29 mcg, Rebif at about 11-21 mcg, or Betaseron at about 0.125-0.24 mg.

39. A method for reducing IL-10, IL-4, IFNγ, TFNα, and/or IL-12p70w levels in a mammal afflicted with an infiltrating immune system response, comprising administering to the mammal a therapeutic amount of an interferon beta.

40. The method of claim 39, wherein the beta-interferon is interferon-β 1a or interferon-β 1b.

41. The method of claim 39, wherein the nervous system inflammation results from multiple sclerosis.

42. The method of claim 39, wherein the beta-interferon is selected from the following or the active ingredient therein: Avonex at a dosage of about 30 mcg once a week, Rebif at a dosage of about 22-44 mcg three times a week, or Betaseron at a doasage of about 0.25 mg every other day.

43. The method of claim 39, wherein the beta-interferon is selected from the following or the active ingredient therein: Avonex at about 15-29 mcg, Rebif at about 11-21 mcg, or Betaseron at about 0.125-0.24 mg.

44. A medicament for use in treating an neurodegenerative disease, the medicament comprising a therapeutic amount of at least one of an estrogen receptor beta ligand and a beta interferon.

45. The medicament of claim 44, wherein the beta-interferon is interferon-β 1a or interferon-β 1b.

46. The medicament of claim 44, wherein the neurodegenerative disease is multiple sclerosis.

47. The medicament of claim 44, wherein the beta-interferon is selected from the following or the active ingredient therein: Avonex at a dosage of about 30 mcg once a week, Rebif at a dosage of about 22-44 mcg three times a week, Betaseron at a doasage of about 0.25 mg every other day.

48. The medicament of claim 44, wherein the beta-interferon is selected from the following or the active ingredient therein: Avonex at about 15-29 mcg, Rebif at about 11-21 mcg, Betaseron at about 0.125-0.24 mg.

49. The medicament of claim 44, wherein the estrogen receptor beta ligand is diarylpropionitrile or estriol selected at a dose of: about 2-16 mg/kg/day, or about 4-12 mg/kg/day, or about 8 mg/kg/day.

50. A medicament for use to limit firm adhesion and/or transendothelial migration of effector cells into the CNS in neurodegenerative disease, the medicament comprising a therapeutic amount of at least one of an estrogen receptor beta ligand and a beta interferon.

51. The medicament of claim 50, wherein the beta-interferon is interferon-β 1a or interferon-β 1b.

52. The medicament of claim 50, wherein the neurodegenerative disease is multiple sclerosis.

53. The medicament of claim 50, wherein the beta-interferon is selected from the following or the active ingredient therein: Avonex at a dosage of about 30 mcg once a week, Rebif at a dosage of about 22-44 mcg three times a week, Betaseron at a doasage of about 0.25 mg every other day.

54. The medicament of claim 50, wherein the beta-interferon is selected from the following or the active ingredient therein: Avonex at about 15-29 mcg, Rebif at about 11-21 mcg, Betaseron at about 0.125-0.24 mg.

55. The medicament of claim 50, wherein the estrogen receptor beta ligand is diarylpropionitrile or estriol selected at a dose of: about 2-16 mg/kg/day, or about 4-12 mg/kg/day, or about 8 mg/kg/day.
